(19) United States
(12) Patent Application Publication
(10) Pub. No.: US 2011/0263946 A1
(43) Pub. Date: Oct. 27, 2011

(54) METHOD AND SYSTEM FOR REAL-TIME AND OFFLINE ANALYSIS, INFERENCE, TAGGING OF AND RESPONDING TO PERSON(S) EXPERIENCES

(75) Inventors: Rana el Kaliouby, Cambridge, MA (US); Rosalind W. Picard, Newtownville, MA (US); Abdelrahman N. Mahmoud, Cairo (EG); Youssef Kashef, Cairo (EG); Miriam Anna Rimm Madsen, Newtown, MA (US); Mina Mikhail, Cairo (EG)

(73) Assignee: MIT Media Lab

(21) Appl. No.: 12/765,555

(22) Filed: Apr. 22, 2010

(51) Int. Cl. A61B 5/00

(52) U.S. Cl. 600/300

(57) ABSTRACT

A digital computer and method for processing data indicative of images of facial and head movements of a subject to recognize at least one of said movements and to determine at least one mental state of said subject is provided. The outputting instructions for providing to a user information relating to at least one said mental state. A further processing data reflective of input from a user, and based at least in part on said input, confirming or modifying said determination and generating with a transducer an output of humanly perceptible stimuli indicative of said at least one mental state.

### BACKGROUND

1. Field of the Disclosed Embodiments

The disclosed embodiments relate to a method and system for real-time and offline analysis, inference, tagging of and responding to person(s) experiences.

2. Brief Description of Earlier Developments

The human face provides an important, spontaneous channel for the communication of social, emotional, affective and cognitive states. As a result, the measurement of head and facial movements, and the inference of a range of mental states underlying these movements are of interest to numerous domains, including advertising, marketing, product evaluation, usability, gaming, medical and healthcare domains, learning, customer service and many others. The Facial Action Coding System (FACS) (Ekman and Friesen 1977; Hager, Ekman et al. 2002) is a catalogue of unique action units (AUs) that correspond to each independent motion of the face. FACS enables the measurement and scoring of facial activity in an objective, reliable and quantitative way, and is often used to discriminate between subtle differences in facial motion. Typically, human trained FACS-coders manually score pre-recorded videos for head and facial action units. It may take between one to three hours of coding for every minute of video. As such, it is not possible to analyze the videos in real-time nor adapt a system's response to the person's facial and head activity during an interaction scenario and while FACS provides an objective method for describing head and facial movements, it does not depict what the emotion underlying those action units are, and says little about the person's mental or emotional state. Even when AU's are used to map to emotional states, these are typically only the limited set of basic emotions, which include happiness, sadness, disgust, anger, surprise and sometimes contempt. Facial expressions that portray other states are much more common in everyday life. Here, facial expressions related to affective and cognitive mental states such as confusion, concentration and worry are far more frequent than the limited set of basic emotions—in a range of human-human and human-computer interaction. The facial expressions of the six basic emotions are often posed (acted) so are depicted in an exaggerated and prototypic way, while, natural, spontaneous facial expressions are often subtle, fleeting and asymmetric, and co-occur with abrupt head movements. As a result, systems that only identify the six prototypic facial expressions have very limited use in real-world applications as they do not consider the meaning of head gestures when making an inference about a person's affective and cognitive state from their face. In existing systems, only a limited set of facial expressions are modeled by assuming a one to one mapping between a face and an emotional state. One to one mapping is very limiting as the same expression can communicate more than one affective and cognitive state and only single, isolated or pre-segmented facial expression sequences are typically considered. Additionally, in applications where real-time feedback of the system based on user state is a requirement, offline manual human coding will not suffice. Even in offline applications, human coding is extremely labor and time intensive and is therefore occasionally used. Accordingly, there is a desire for automatic and real-time methods.

### SUMMARY OF THE EXEMPLARY EMBODIMENTS

In accordance with one exemplary embodiment, a method is provided, with a digital computer processing data indicative of images of facial and head movements of a subject to recognize at least one of said movements and to determine at least one mental state of said subject. The outputting instructions for providing to a user information relating to at least one said mental state. A further processing data reflective of input from a user, and based at least in part on said input, confirming or modifying said determination and generating with a transducer an output of humanly perceptible stimuli indicative of said at least one mental state.

In accordance with another exemplary embodiment a method is provided, with a digital computer processing data indicative of images of facial and head movements of a subject to determine at least one mental state of said subject and associating the at least one mental state with at least two events, wherein at least one of said events is indicated by said data indicative of images of facial and head movements. The at least one other of said events is indicated by another data set, which other data set comprises content provided to said subject or data recorded about said subject.

In accordance with yet another exemplary embodiment, an apparatus is provided having the at least one camera for capturing images of facial and head movements of a subject. At least one computer is adapted for analyzing data indicative of said images and determining one or more mental states of said subject, and outputting digital instructions for providing a user substantially real time information relating to said at least one mental state. The computer is adapted for analyzing data reflective of input from a user, and based at least in part on said user input data analysis, changing or confirming said determination.

In accordance with yet another exemplary embodiment, an article of manufacture comprising a machine-accessible medium is provided having instructions encoded thereon for enabling a computer to perform the operations of processing data indicative of images of facial and head movements of a subject to recognize at least one said movement and to determine at least one mental state of said subject. The encoded instructions on the medium enable the computer to perform outputting instructions for providing to a user information relating to said at least one mental state and processing data reflective of input from a user, and based in least in part on said input, confirm or modify said determination.

### BRIEF DESCRIPTION OF THE DRAWINGS

The foregoing aspects and other features of the exemplary embodiments are explained in the following description, taken in connection with the accompanying drawings, wherein:

FIGS. 1A-1C are respectively isometric views of several exemplary embodiments of a method and system;

FIG. 2 is a system architecture diagram;

FIG. 3 is a time analysis diagram;

FIG. 4 is a flow chart;

FIG. 5 is a flow chart;

FIGS. 6A-6B are flow charts respectively illustrating different features of the exemplary embodiments;

FIGS. 7-7A are flow charts respectively illustrating further features of the exemplary embodiments;

FIG. 8 is a flow chart;

FIG. 9 is a graphical representation of a head and facial activity example;

FIG. 10 is another graphical representation of a head and facial activity example;

FIG. 11 is a schematic representation of person's face;

FIG. 12 is a flow chart;

FIG. 13 is a flow chart;

FIG. 14 is a flow chart;

FIG. 15 is a flow chart;

FIG. 16 is a flow chart;

FIG. 17 is a user interface;

FIG. 18 is a flow chart;

FIG. 19 is a log file;

FIG. 20 is a system interface;

FIG. 21 is a system interface;

FIG. 22 is a system interface;

FIG. 23 is a system interface; and

FIG. 24 is a bar graph.

### DETAILED DESCRIPTION OF THE EXEMPLARY EMBODIMENT(S)

As will be described below, the disclosed embodiments relate to a method and system for the automatic and semi-automatic, real-time and offline, analysis, inference, tagging of head and facial movements, head and facial gestures, and affective and cognitive mental states from facial video, thereby providing important information that yields insight related to people's experiences and enables systems to adapt to this information in real-time. Here, the system may be selectable between what may be referred to as a assisted or semi-automatic analysis mode (as will be described further below) and an automatic analysis mode. The disclosed embodiments may utilize methods, apparatus' or subject matter disclosed in the University of Cambridge Technical Report Number 636 entitled Mind Reading Machines: Automated Inference of Complex Mental States dated July 2005 and having UCAM-CL-TR-636 and ISSN 1476-2986 which is hereby incorporated by reference herein in its entirety. Although the disclosed embodiments will be described with reference to the embodiments shown in the drawings, it should be understood that the present invention can be embodied in many alternate forms of embodiments. In addition, any suitable size, shape or type of elements or materials could be used.

With respect to the disclosed embodiments, the phrase “real-time” analysis refers to that head and facial analysis is performed on a live feed from a camera, on the go during an interaction, enabling the system to respond to the person's affective and cognitive state. The phrase “offline” analysis refers to that head and facial analysis performed on pre-recorded video. The phrase “automatic” analysis refers to that head and facial analysis is done completely by the machine without the need for a human coder. The phrase “assisted” analysis and inference refers to the head and facial analysis and related inference (such as mental state inference, event inference and/or event tagging or relating with one or more head and facial activity and/or mental states) performed by the machine with input from a human observer/coder. The phrase “feature points” means identified locations on the face that define a certain facial area, such as the inner eye brow or outer eye corner. The phrase “action unit” means contraction or other activity of a facial muscle or muscles that causes an observable movement of some portion of the face. These can be derived by observing static or dynamic images. The phrase “motion action units” refers to those head action units that describe head and facial movements and can only be calculated from video or from image sequences. The phrase “gesture” means head and/or facial events that have meaning potential in the contexts of communication. They are the logical unit that people use to describe facial expressions and to link these expressions to mental states. For example, when interacting with a person whose head movement alternates between a head-up and a head-down action, with a certain range of frequency and duration, most people would abstract this movement into a single event [e.g., a head nod]. The phrase “mental state” refers collectively to the different states that people experience and attribute to each other. These states can be affective and/or cognitive in nature. Affective states include the emotions of anger, fear, sadness, joy and disgust, sensations such as pain and lust, as well as more complex emotions such as guilt, embarrassment and love. Also included are expressions of liking and disliking, wanting and desiring, which may be subtle in appearance. These states could also include states of flow, discovery, persistence, and exploration. Cognitive states reflect that one is engaged in cognitive processes such as thinking, planning, decision-making, recalling and learning. For instance, thinking communicates that one is reasoning about, or reflecting on some object. Observers infer that a person is thinking when his/her head orientation and eye-gaze is directed to the left or right upper quadrant, and when there is no apparent object to which their gaze is directed. Detecting thinking state is desired because, depending on the context, it could also be a sign of disengagement, distraction or a precursor to boredom. Confusion communicates that a person is unsure about something, and is relevant in interaction, usability and learning contexts. Concentration is absorbed meditation and communicates that a person may not welcome interruption. Cognitive states also include self-projection states such as thinking about the upcoming actions of another person, remembering past memories, or imagining future experiences. The phrase “analysis” refers to methods that localize and extract various texture and temporal features that describe head and facial movements. The phrase “inference” and “inferring” refer to methods that are used to compute the person's current affective and cognitive mental state, or probabilities of several such possible states, by combining head and facial movements starting sometime in the past up to the current time, as well as combining other possible channels of information recorded alongside or known prior to the recording. The phrase “tagging” or “indexing” refers to person-based or machine-based methods that mark a person's facial video or video of the person's field of vision (what the person was looking at or interacting with at the time of recording) with points of interest (e.g., marking when a person showed interest or confusion). The phrase “prediction” refers to methods that consider head and facial movements starting sometime in the past up to the current time, to compute the person's affective and cognitive mental state sometime in the future. These methods may incorporate additional channels of past information. The phrase “intra-expressions dynamics” refers to the temporal structure of facial actions within a single expression. The phrase “inter-expression dynamics” refers to the temporal relation or the transition in time, between consecutive head gestures and/or facial expressions.

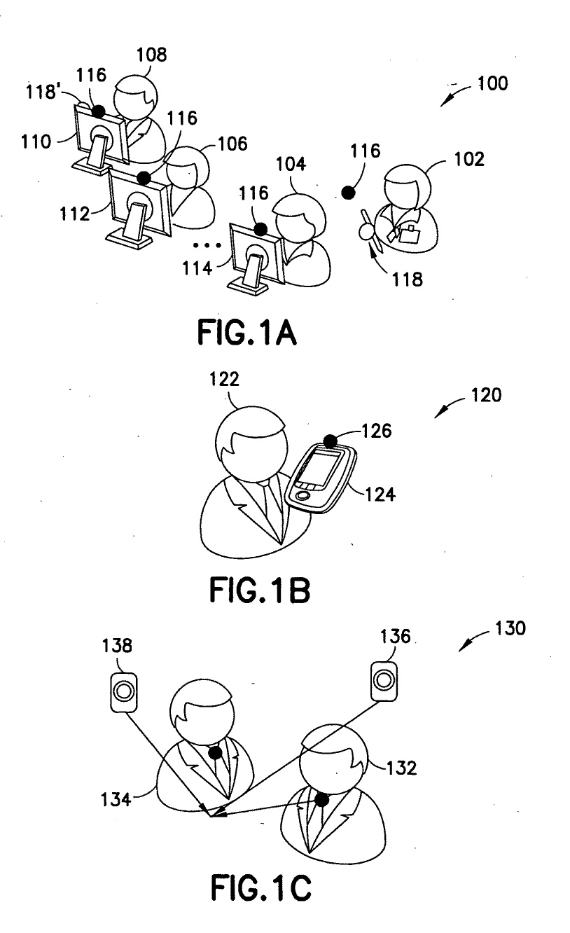

Referring now to FIGS. 1A-1C, there are shown several exemplary embodiments of the method and system. In the embodiment 100 of FIG. 1A, one or more persons 102, 104, 106, 108 are shown viewing an object or media on a display such as a monitor, or TV screen 110, 112, 114 or engaged in interactive situations such as online or in-store shopping, gaming. By way of example, a person is seated in front (or other suitable location) of what may be referred to for convenience in the description as a reader of head and facial activity, for example a video camera 116, while engaged in some task or experience that include one or more events of interest to the person. Camera 116 is adapted to take a sequence of image frames of a face of the person during an event during the experience the camera where the sequence may be derived where the camera is continually recording during the experience. An “experience” may include one or more persons passive viewing of an event, object or media such as watching an advertisement, presentation or movie, as well as interactive situations such as online or in-store shopping, gaming, other entertainment venues, focus groups or other group activities; interacting with technology (such as with an e-commerce website, customer service website, search website, tax software, etc), interacting with one or more products (for example, sipping different beverages that are presented to the person) or objects over the course of a task, such as trying out a new product, e-learning environment, or driving a vehicle. The task may be passive such as watching an advertisement on a phone or other electronic screen, or immersive such as evaluating a product, tasting a beverage or performing an online task. For example, a number of participants (e.g. 1-35 or more) may be seated in front of Macbook™ laptops with built-in iSight™ cameras and recorded while repeatedly sampling different beverages. In an alternate example, in an exhibition set up, participants walk up to a large monitor which has a Logitech camera located on the top or bottom of the monitor. In alternate embodiments, the camera may be used independent of a monitor, where, for example, the event or experience is not derived from the monitor. In the exemplary embodiment, one or more video cameras 116 record the facial and head activity of one or more persons while undergoing an experience. The disclosed embodiments are compatible with a wide range of video cameras ranging from inexpensive web cams to high-end cameras and may include any built-in, USB or Firewire camera that can be either analog or digital. Examples of video equipment include an Hewlett Packard notebook built-in camera (1.3 Mega Pixel, 25 fps), iSight for macs (1.3 Mega Pixel, 30 fps), Sony Vaio™ built-in camera, Samsung Ultra Q1™ front and rear cameras, Dell built-in camera, Logitech cameras (such as Webcam Pro 9000™, Quickcam E2500™, Quickfusion™), Sony camcorders, Pointgrey firewire cameras (DragonFly2, B&W, 60 fps). Alternately, analog, wireless cameras in combination with an analog-to-digital converter such as the KWorld Xpert DVD Maker USB 2.0 Video Capture Device, which captures videos at 30 frames per second. The disclosed embodiments performs at 25 frames per second and above, but may also functions at lower frame rates, for example, 5 frames per second. In alternate embodiments more or less frames per second may be provided. The disclosed embodiments may utilize camera image resolutions between 320×240 to 640×480. While lower resolutions degrade the accuracy of the system, higher or lower resolution images may alternately be provided. In the disclosed embodiments, the person's field of vision (what the person is looking at) may also be recorded, for example with an eye tracker. This could be what the person is viewing on any of a computer, a laptop, other portable devices such as camera phones, large/small displays such as those used in advertising, TV monitors. In these cases a screen capture system may be used to capture the person's field of view, for example, a TechSmith Screen capture. The object of interest may be independent of a monitor, such as where the object of interest may also be other persons or other objects or products. In these cases an external video camera that points at the object of interest may be used. Alternatively, a camera that is wearable, on the body and points outwards can record the person's field of view for situations in which the person is mobile. Alternately, multiple stationary or movable cameras may be provided and the images sequenced to track the person of interest and their facial features and gestures. Interactions of a person may include passive viewing of an object or media such as watching an advertisement, presentation or movie, as well as interactive situations such as online or in-store shopping, gaming, other entertainment venues, focus groups or other group activities; interacting with one or more products or objects over the course of a task, such as trying out a new product, driving a vehicle, e-learning; one or more persons interacting with each other such as students and student/teacher interaction in classroom-based or distance learning, sales/customer interactions, teller/bank customer, patient/doctor, parent/child interactions; interacting with technology (such as with an e-commerce website, customer service website, search website, tax software, etc). Here, interactions of a person may include any type of event or interaction that elicits a affective or cognitive response from the person. These interactions may also be linked to factors that are motivational, providing people with the opportunity to accumulate points or rewards for engaging with such services.

The disclosed embodiments may also be used in a multi-modal setup jointly with other sensors 118 including microphones to record the person's speech, physiology sensors to monitor skin conductance, heart rate, heart rate variability and other suitable sensors where the sensor senses a physical state of the person's body. For example, microphones may include built-in microphones, wearable microphones (e.g., Audio Technica AT892) or ambient microphones. Alternately a camera may have a built-in microphone or otherwise. The physiology sensors may include a wearable and washable sensor for capturing and wirelessly transmitting skin conductance, heart rate, temperature, and motion information such as disclosed in U.S. patent application Ser. No. 12/386,348 filed Apr. 16, 2009, which is hereby incorporated by reference herein in its entirety. Further, it is also possible to use the system in conjunction with other physiological sensors. In a multi-modal set up, participants are asked to wear these sensors as well as recording their face and field of vision, while engaging in an interaction. The data from tagged interactions or events and from the video equipment as well as the sensors are synchronized, visualized and used with multi-modal algorithms to infer affective and cognitive states of interest correlated to the events or interactions. Data from multiple streams may provide redundancy therefore increasing confidence in a given inference, complementary (for example, the face gives valence information, whereas physiology yields important arousal information or otherwise), contradictory (for example, when voice inflection is inconsistent with face communications). The system may further be used with an eye tracker 118′, where the eye tracker is adapted to track a location where the person is gazing, with an event occurring at the location and the location stored upon occurrence of the event and tagged with the event of the experience. Here, the location may be stored upon occurrence of the event and tagged with the event and the mental state inferred based on a particular action of interest occurring at the location. Here, the gaze location being registered upon occurrence of the event at a location and tagged with the event and the mental state inferred upon occurrence of the event when the gaze location is substantially coincident with a location. The eye tracker identifies where the person is looking, whatever is displayed, for example, a monitor is recorded to give the event of interest, or by way of further example, an activity may be recorded. These two things may be combined with the face-analysis system and to inferred the person's state when they were looking at something in particular or of particular interest.

In the embodiment 120 of FIG. 1B, one or more persons 122 are shown viewing an object or media on cell phone 124 facial video recorded using a built-in camera 126 in phone 124. Here, a person 122 is shown using their portable digital device (e.g., netbook), or mobile phone (e.g., camera phones) or other small portable device (e.g., iPOD) and is interacting with some software or watching video. In the disclosed embodiment, the system may run on the digital device or alternately, the system may run networked remotely on another device.

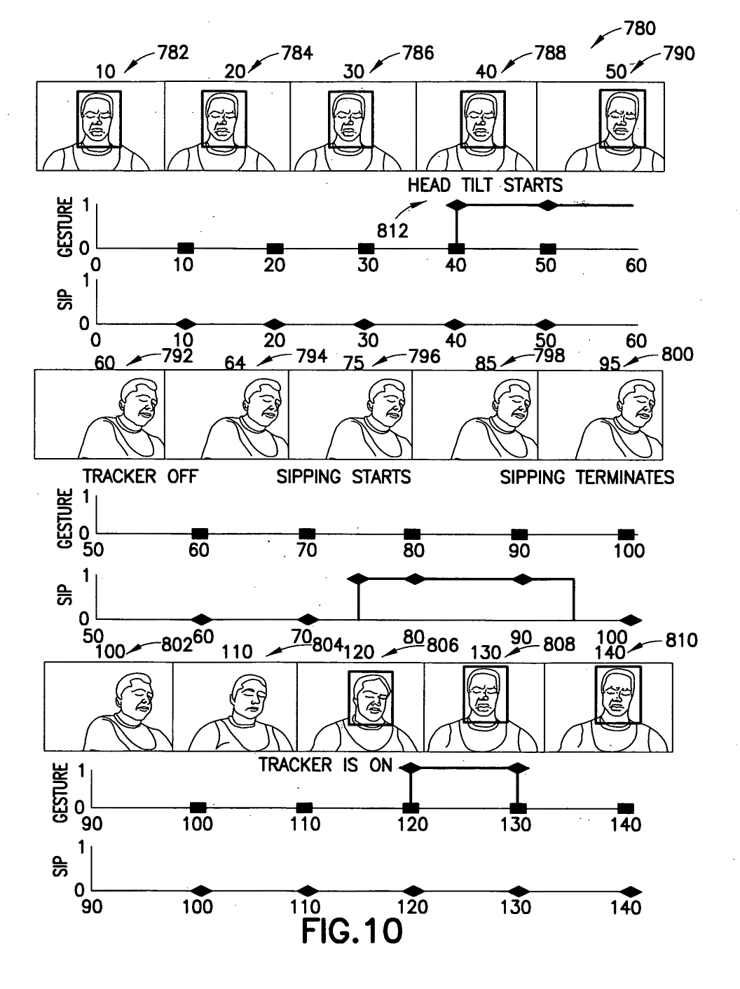

In embodiment 130 of FIG. 10, one or more persons 132, 134 are shown in a social interaction with other people, robots, or agent. Cameras 136, 138 may be wearable and/or mounted statically or moveable in the environment. In embodiment 130, one or more persons are shown interacting with each other such as students and student/teacher interaction in classroom-based or distance learning, sales/customer interactions, teller/bank customer, patient/doctor, parent/child interactions. In alternate embodiments any suitable interaction may be provided. Here, one or more persons in a social interaction with other people, robots, or agents have cameras, or other suitable readers of head and facial activity, that may be wearable and/or mounted statically or movable within the environment. As an example, the system may be running on an ultra mobile device (Samsung Ultra Q1) which has a front and rear-facing camera. A person, holding up the device, would record and analyze his/her interaction partner as they go about their social interactions. In the embodiments of FIGS. 1A-1C, the person is free to move about naturally as long as at least half of their face can be seen by the camera. As such, where people do not have to restrict their head movement and keep from touching their face during the session is within the scope of the disclosed embodiments. The apparatus constitutes one or more video cameras that record one or more person's facial and head activity as well as one or more person's field of vision (what the person(s) are looking at), which could be on a computer, a laptop, other portable devices such as camera phones, large/small displays such as those used in advertising, TV monitors, or whatever other object the person is looking at. The cameras may also be wearable, worn overtly or covertly on the body. The video camera may be a high-end video camera, as well as a standard web camera, phone camera, or miniature high-frame rate or other custom camera. By way of example, the video camera may include an eye tracker for tracking a persons gaze location, or otherwise gaze location tracking may be provided with any other suitable means. The video camera may be mounted on a table immediately behind a monitor on which the task will be carried out; it may also be embedded in the monitor and/or cell phone, or wearable. A computer (desktop, laptop, other portable devices such as the Samsung Ultra Q1) runs one instance of the system. In alternate embodiments, multiple instances of the system may be run on one or more devices and networked where the data may be aggregated. By way of further example. in alternate embodiments, one instance may be run on a device and the data from multiple cameras and people may be networked to the device where the data may be processed and aggregated.

As will be described in greater detail below, the disclosed embodiments 100, 120, 130 relate to a method and system for 1) automatic real-time or offline analysis, inference, indexing, tagging, and prediction of people's affective and cognitive experiences in a variety of situations and scenarios that include both human-human and human-computer interaction contexts; 2) real-time visualization of the person's state, as well as real-time feedback and/or adaptation of a system's responses based on one or more person's affective, cognitive experiences; 3) assisted real-time analysis and tagging where the system makes real-time inferences and suggestions about a person's affective and cognitive state to assist a human observer with real-time tagging of states, and 4) assisted offline analysis and indexing of events, that is combined with the tagging of one or more human observers to improve confidence in the interpretation of the facial-head movements; 5) assisted feedback and adaptation of an experience or task to a person's inferred state; 6) offline aggregation of multiple person's states and its relation to a common experience or task.

The disclosed embodiments utilize computer vision and machine learning methods to analyze incoming video from one or more persons, and infer multiple descriptors, ranging from low-level features that quantify facial and head activity to valence tags (for example, positive, negative, neutral or otherwise), affective or emotional tags (for example, interest, liking, disliking, wanting, delight, frustration or otherwise), and cognitive tags (for example, cognitive overload, understanding, agreement, disagreement or otherwise), and memory indices (for example, whether an event is likely to be memorable or not or otherwise). The methods combine bottom-up vision-based processing of the face and head movements (for example, a head nod or smile or otherwise) with top-down predictions of mental state models (for example, interest and agreeing or otherwise) to interpret the meaning underlying head and facial signals over time.

As will be described below, a data-driven, supervised, multilevel probabilistic Bayesian model handles the uncertainty inherent in the process of attributing mental states to others. Here, the Bayesian model looks at channels observed and infers a hidden state. The data-driven model trains new action units, gestures or mental states with examples of these states, such as several videos clips portraying the state or action of interest. Here, the algorithm is generic and is not specific to any given state, for example, not specific to liking or confusion. Here, the same model is used, but may be trained for different states and end up with a different parameter set per state. This model is in contrast with non data-driven approaches where, for each new state, an explicit function or method has to be programmed or coded for that state. Provided clear examples of a state, data-driven methods are in general more scalable.

The disclosed embodiments utilize inference of affective and cognitive states including and extending beyond the basic emotions and relating low-level features that quantify facial and head activity with higher level affective and cognitive states as a many-to-many relationship, thereby recognizing that 1) a single affective or cognitive state is expressed through multiple facial and head activities and 2) a single activity can contribute to multiple states. Here, the multiple states may occur simultaneously, overlap or occur in sequence. The edges and weights between a single activity and a single state are inferred manually or by using machine learning and feature selection methods. These represent the strength or discriminative power of an activity towards a state. Affective and cognitive states are modeled as independent classifiers that are not mutually exclusive and can co-occur, accounting for the overlapping of states in natural interactions. The disclosed embodiments further utilize a method to handle head gestures in combination with facial expressions and a method to handle inter- and intra-expression dynamics. Affective and cognitive states are modeled such that consecutive states need not pass through neutral states. The disclosed embodiments further utilize analysis of head and facial movements at different temporal granularities, thereby providing different levels of facial information, ranging from low-level movements (for example, eyebrow raise or otherwise) to a depiction of the person's affective and cognitive state. The disclosed embodiments may utilize automatic, real-time analysis or selectably utilize a real time, assisted analysis with human facial coder(s).

The disclosed embodiments further relate to a method of real-time and or offline analysis, inference, tagging and feedback method that presents output information beyond graphs—e.g. summarizing features of interest (for example, such as frowns or nose wrinkles or otherwise) as bar graphs that can be visually compared to neutral or positive features (for example, such as eyebrow raises or smiles involving only the zygomate or otherwise), mapping output to LED, sound or vibration feedback in applications such as conversational guidance systems and intervention for autism spectrum disorders. In alternate embodiments, any suitable indication of state may be provided either visual by touch or otherwise. The disclosed embodiments further relate to a method for real-time visualization of a person's affective-cognitive states as well as a method to compute aggregate or highlights of a person's state in response to an event or experience (for example, the highlights of a show or video are instantly extracted when viewers smile or laugh, and those are set aside and used for various purposes or otherwise). The disclosed embodiments further relate to a method for the real-time analysis of head and facial analysis movements and real-time action handlers, where analyses can trigger actions such as alerts that trigger display of an empathetic agent's face (for example, to show caring/concern to a person who is scowling or otherwise). The disclosed embodiments further relate to a method and system for the batch offline analysis of head and facial activity in video files, and automatic aggregation of results over the course of one video (for example, one participant) as well as across multiple persons. The disclosed embodiments further relate to a method for the use of recognized head and facial activity to identify events of interest, such as a person sipping a beverage, or a person filling an online questionnaire, fidgeting or other events that, are pertinent to specific applications. The disclosed embodiments further relate to a method and system for assisted automatic analysis, combining real-time analysis and visualization or feedback regarding head and facial activity and/or mental states, with real-time tagging of states of interest by a human observer. The real-time automatic analysis assists the human observer with the real-time tagging. The disclosed embodiments further relate to a method and system for assisted analysis, for combining human observer input with real time automatic machine analysis of facial and head activity to substantially increase accuracy and save time on the analysis. For example, a system makes a guess, passes to one or more persons (who may be remote one from the other), combines their inputs in real time and improves the system's accuracy while contributing to an output summary of what was found and how reliable it was. The disclosed embodiments further relate to a method and system for assisted analysis, using automated analysis of head and facial activity. For instance, manually coding videos in a conventional manner for facial expressions or affective states may take a coder on average 1 hour for each minute of video. Typically, at least 2 or 3 coders are needed using the conventional approach to establish validity of the coding, resulting in many hours of coding a very labor-intensive and time-consuming approach. The disclosed embodiments further relate to a method for supervised, texture-based action unit detection that uses fiducial landmarks to define regions of interest that are the center of Gabor jets. This approach allows for training new action units, supporting action units that are texture-based, runs automatically and in real-time. The disclosed embodiments further relate to a method and system for retraining of existing and training of new action units, gestures and mental states requiring only short video exemplars of states of interest. The disclosed embodiments further relate to a method to combine information from the face with other channels (including but not limited to head activity, body movements, physiology, voice, motion) and contextual information (including but not limited to task information, setting) to enhance confidence of an interpretation of a person's state, as well as extend the range of states that can be inferred. The disclosed embodiments further relate to a method whereby interactions can also be linked to factors that are motivational, providing people with the opportunity to accumulate points or rewards for engaging with such services.

The disclosed embodiments further relate to a method and system for the real-time or offline measurement and quantification of people's affective and cognitive experiences from video of head and facial movements, in a variety of situations and scenarios. The person's affective, cognitive experiences are then correlated with events and may provide real-time feedback and adaptation of the experience, or the analysis can be done offline and may be combined with a human observer's input to improve confidence in the interpretation of the facial-head movements.

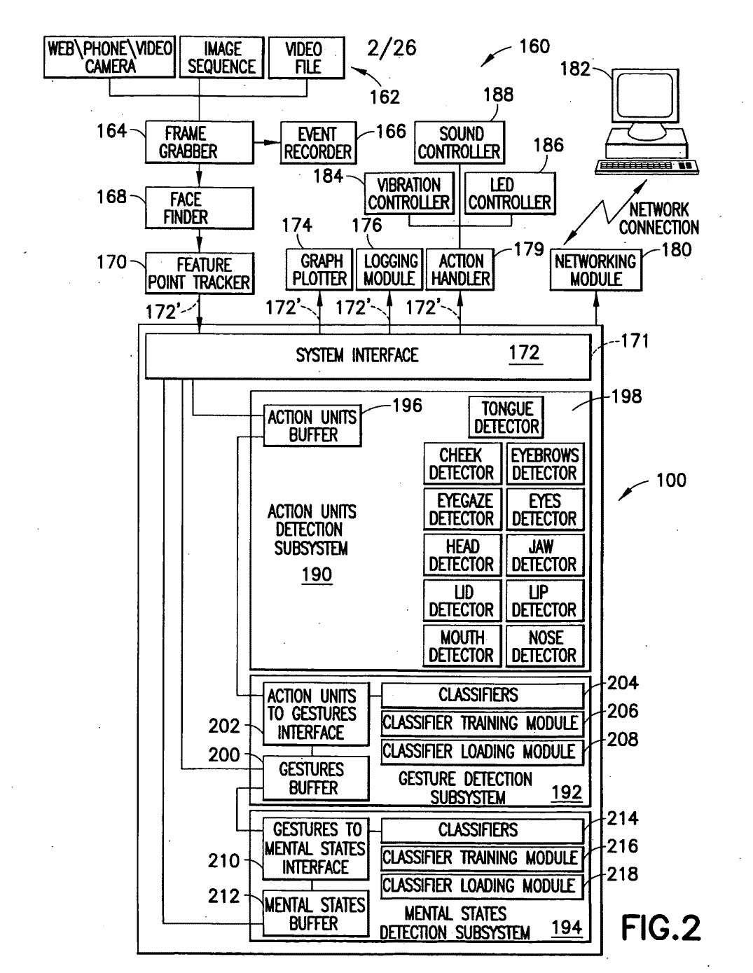

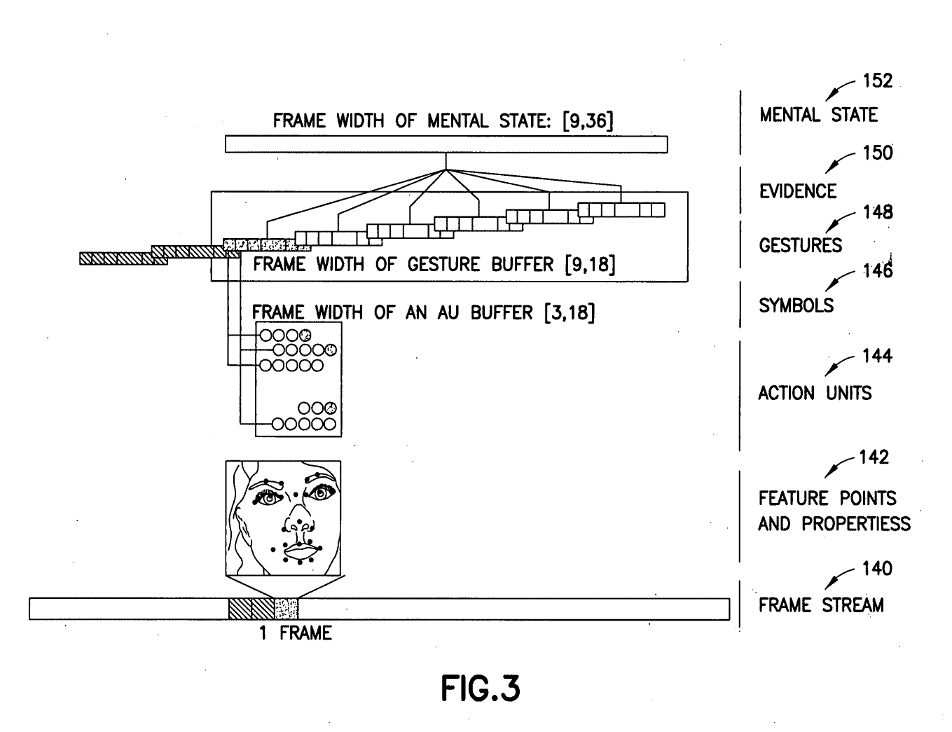

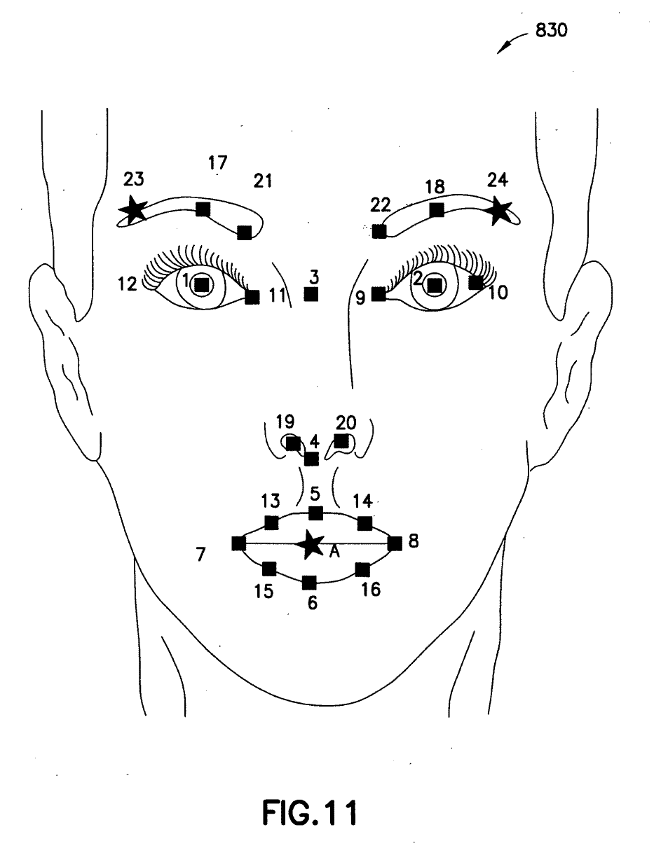

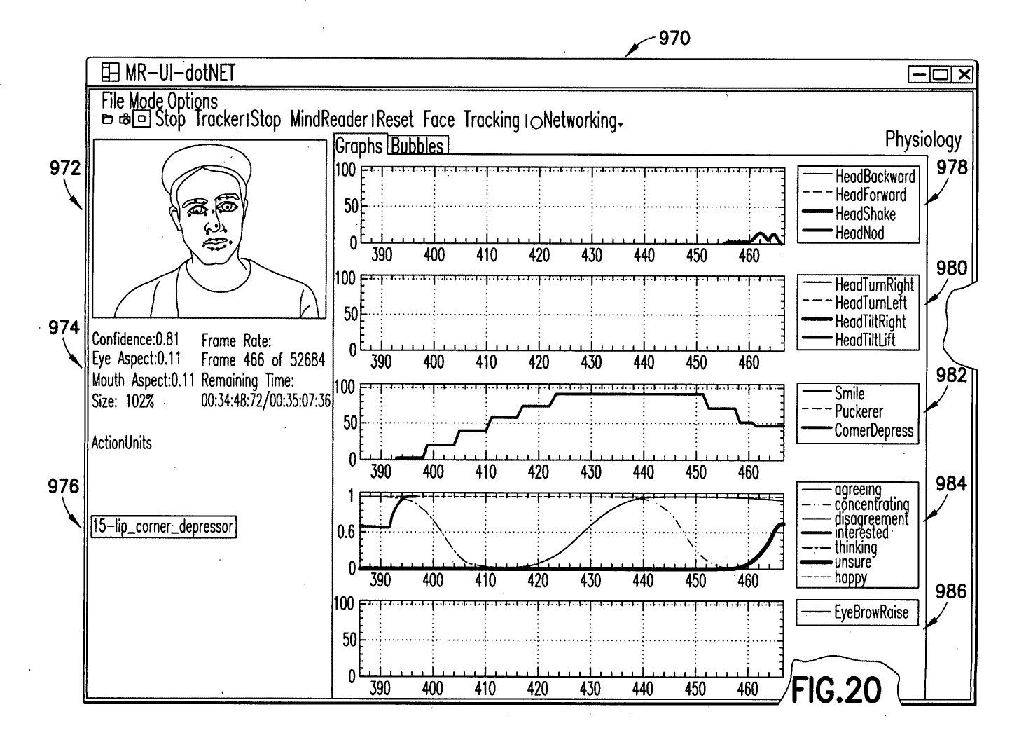

Referring now to FIG. 2, there is shown a schematic block diagram illustrating the general architecture and the functionality of system 100. Although the components of system 100 are shown interconnected as a system, in alternate embodiments, the components may be interconnected in many different ways and more or less components may be provided. In addition, components of system 100 may be run on one or more multiple platforms, where networking may provided for server aggregation where the results from different machines and processing may provide for aggregate analysis with the networking. Referring also to FIG. 3, there is shown a graphical representation of a temporal analysis performed by system 100. The person's facial expressions and head gestures are recorded in frame stream 140 during the interaction where the frame stream has a stream of frames recorded during events or interactions of interest. The frames are analyzed in real-time or recorded and/or analyzed offline where feature points and properties 142 of the face are detected. Here, the system has an electronic reader 162 (see also FIGS. 1A-1C) that obtains facial and head activity data from the person experiencing a event of an experience. In the exemplary embodiment, an event recorder is connected to the reader and may be configured for registering the occurrence of the event, such as from the data obtained from the reader. Accordingly, the system may automatically recognize and register the event from the facial and head activity data obtained by the reader. In alternate embodiments, the event recorder may be configured to recognize and register the occurrence of the event of interest from any other suitable data transmitted to the event recorder. The system 100 may further automatically infer from the facial and head activity data obtained by the reader a head and facial activity descriptor (e.g. action units 144, see also FIG. 3) 190 of a head and facial act of the person. The system takes the feature points and properties 142 within the frames and may for example derive action units 144, symbols 146, gestures 148, evidence 150 and mental states 152 from individual and sequences of frames. In the embodiment shown, the system has a head and facial activity detector 190 connected to the reader and configured for inferring from the reader data a head and facial activity descriptor of a head and facial activity of the person. Here, the system may for example automatically infer from the head and facial activity descriptor data a gesture descriptor of the face, the gesture descriptor being inferred dynamically from the head and facial activity descriptor. In the embodiment shown, the system may also have a gesture detector 192 connected to the head and facial activity detector 190 and configured for dynamically inferring a gesture descriptor of the head and facial activity of the person using for example the head and facial activity descriptor or directly from the reader data without head and facial activity descriptor data from the head and facial activity detector. The system has a mental state detector 194 connected to the reader 162 and configured for dynamically inferring the mental state from the reader data. In the exemplary embodiment shown, the gesture detector 192 and the head and facial activity detector 190 may input gesture descriptor and head and facial activity descriptor data (e.g. data defining gestures 148, symbols 146 and/or action units 142) to the mental states detector 194. The mental states detector may infer one or more mental states using one or more of the gesture descriptor and head and facial activity descriptor data, The mental states detector 194 may also infer mental states 152 directly from the head and facial activity data from the reader 162 without input or data from the gesture and/or head and facial activity detectors 190, 192. The system dynamically infers the mental state(s) of the person and automatically generates a predetermined action in action handler 178 related to the event in response to the inferred mental state of the person. In the exemplary embodiment, the mental states detector, the gestures detector and head and facial activity detector are shown as discrete units or modules of system 100, for example purposes. In alternate embodiments, the mental states detector may be integrated with the head and facial activity detector and/or gestures detector in a common integrated module. Moreover in other alternate embodiments, the system may have a mental states detector connected to the reader without intervening head and facial activity detector(s) and/or gestures detector(s). Action handler 178 may generate a predetermined action that is a user recognizable indication of the mental state, generated by the action handler or generator on an output device in substantial real time with the occurrence of the event. Here, going from action units (AU) to gestures and from AU's and gestures to mental states involves dynamic models where the system puts into consideration a temporal sequence of AU's to infer a gesture. The results of the analysis are provided in the form of log files as well as various visualizations as described below with regard to the “Action Handler” and by way of example in FIGS. 20-24. Here, an action generator 178 is provided connected to the mental state detector and configured for generating, substantially in real time, a predetermined action related to the event in response to the mental state. Referring back to FIGS. 2 and 3, the system architecture 160 consists of either a pre-recorded video file input or a video camera or image sequence 162 the data from which is fed to the system via the system interface 172 in substantially real-time with occurrence of the event. In the event frame grabber 164 is utilized for a video (an image sequence), one frame is automatically extracted at a time (at recording speed). The video or image sequence may be recorded or captured in real time. Multiple streams of video or image sequences from multiple persons and events may further be provided. Here, Multi modal analysis may be provided where single or multiple instances of the software may be running networked to multiple devices and data may be aggregated with a server or otherwise. Event recorder 166 may also correlate events with frames or sequences of frames. A video of the person's field of view may also be recorded. Face-finder module 168 is invoked to locate a face within the frame. The status of the tracker, for example, whether a face has been successfully located, provides useful information regarding a person's pose especially when combined with knowledge about the person's previous position and head gestures. By way of example, it is possible to infer that the person is turning towards a beverage on their left or right for a sip. Facial feature tracker 170 then locates a number of facial landmarks on the face. These facial landmarks or feature points are typically located on the eyes and eyebrows for the upper face and the lips and nose for the lower face. One example of a configuration of facial feature points is shown in FIG. 11. In the event that the confidence of the tracker falls below a predefined level, which may occur with sudden large motions of the head, the tracker is re-initialized by invoking the face-finder module before attempting to relocate the feature points. A number of face-trackers and facial feature tracking systems may be utilized. One such system is the face detection function in Intel's OpenCV Library implementing Viola and Jones face detection algorithm [REF]. Here, this function does not include a facial feature detector. The disclosed embodiments may use an off-the-shelf face-tracker, for example, Google's FaceTracker, formerly Nevenvision's facial feature tracking SDK. The face-tracker may use a generic face template to bootstrap the tracking process, initially locating the position of facial land-marks. Template files may have different numbers of feature points; current embodiments include templates that locate 8, 14, or 22 feature points, numbers which could change with new templates. In alternate embodiments, more or less feature points may be detected and or tracked. Groups of feature points are geometrically organized into facial areas such as the mouth, lips, right eye, nose, each of which are associated with a specific set of facial action units. The analytic core (e.g. AU detector 190, gestures detector 192, and mental states detector 194, as well as action generator 179 of the disclosed system architecture and methods may be bundled with or into system interface 172 that can plug into any frame analysis and facial feature tracking system. The system interface 172 may interface with mode selector 171 where the system is selectable between one or more types of assisted analysis wherein the system provides information to a user and accepts input from the user and one or more types of automatic analysis. By way of example, when in a assisted analysis mode wherein the system is configured to provide information to a user and accept inputs from the user, sequences of AU's, gestures and mental states may be analyzed in a real time, or off line, with analysis of facial activity and the mental states by a machine or human observer alone or in combination, and identification and/or, tagging of events with the corresponding AU's, gestures, or other identified read and facial activity descriptors for example, and mental states by a human observer alone or in combination with the processing system. By way of further example, when in an automatic analysis mode, sequences of action units, gestures and mental states may be analyzed wholly by the processor programming with a real time or off line analysis of facial activity and mental states, and real time triggering of actions by action handler 178. In alternate embodiments, any suitable combination of operating modes or types of automatic or assisted inference may be provided or may be selectable.

Still referring to FIG. 2, system interface 172 may further interface externally with graph plotter 174, logging module 176, action handler 178 or networking module 180. In alternate embodiments, system interface 172 may interface with any suitable module or device for analysis and or output of the data relating to the action units, gestures or mental states. In alternate embodiments, modules such as the frame grabber, face finder or feature point tracker or any suitable module may be integrated above or below system interface 172. For example, a face finder may be provided to find a location of a face within a frame. By way of further example, a feature point tracker may be provided where the feature point tracker tracks points of features on the face. Networking module 180 interfaces with one or more client machines 182 via a network connection. In alternate embodiments, multiple instances of one or more modules of the system may interface with a host machine over a network where data from the multiple instances is aggregated and processed. The client machines may be local or remote where the network may be wireless, ethernet, and may utilize the internet or otherwise. The client machines may be in the same room or with persons in different rooms. In alternate embodiments, one or more client machines may have modules of the system running on the client machines, for example camera's, frame grabbers, face finders or otherwise. In the exemplary embodiment shown in FIG. 2, the system interface may include a “plug and play” type connector 172′ (one such connector shown for example purposes, and the interface may have any suitable number of “plug and play” type connectors. The “plug and play” connector 172′ is shown for example as being joined to the system interface, and coupling the processor system to the input devices 164, 168, 170 and output devices 174-188. In alternate embodiments any one or more of the modules or portions of the processor system (e.g. head and facial activity detectors 190, 192, mental state detector 194, action handler 179) may have distinct “plug and play” type connectors enabling the processor system to interface automatically with the various input/output devices of the system 100 upon coupling of said input/output devices to the connector. Networking module 180 may provided for server aggregation where the results from different machines and processing may provide for aggregate analysis with networking. With networking module 180, a system for real time inference of a group of participants experiences may be provided where multiple cameras adapted to take sequences of image frames of the faces of the participants during an event during the experience may be provided. Here, multiple face finders adapted to find locations of the faces in the frames, multiple feature point trackers adapted to track points of features on the faces, and multiple action unit detectors adapted to convert locations of the points to action units, and multiple gesture detectors adapted to convert sequences of action units to sequences of gestures, and multiple mental state detectors adapted to infer sequences of mental states from the action units and the gestures may be provided. The sequences of action units, gestures and mental states may be stored upon occurrence of an event and tagged with the event, where data from the mental states is aggregated and a distribution of the mental states of the participants is compiled. Action generator or handler 178 may interface with vibration controller 184 that maps certain gestures or mental state probabilities to a series of vibrations that vary in duration and frequency to portray different states, for example, to give the person wearing the system real-time feedback as they interact with other persons. The action handler 178 may further interface with LED controller 186 which maps certain gesture or mental probabilities of mental states to a green, yellow or red LED which can be mounted on the frame of an eyeglass or any other wearable or ambient object, for example, to give the person wearing the system real-time feedback as they interact with other persons, for example, green may mean that the conversation is going well, red may mean that the person may need to pause and gauge the interest level of their interaction partner, or sound controller 188, which maps certain gesture or mental state probabilities to pre-recorded sound sequences. In alternate embodiments, action handler 178 may interface with any suitable device to indicate the status of mental states or otherwise. By way of example, a high probability of “confusion” that persists over a certain amount of time may trigger a pre-recorded sound file that informs the person using the system that this state has occurred and may provide advice on the course of action to take, for example, “Your interaction partner is confused; please pause and ask if they need help”. In the exemplary embodiment the action handler 178 may also interface with one or more of the controllers 184-188 to map certain data from other sensors such as physiology sensors 118 (e.g. skin consultant, heart rate) to corresponding display or other output indicia that may be recognized by a user. Networking module 180 may interface with one or more client machines 182. System interface 172 further interfaces with action unit detection subsystem 190, gesture detection subsystem 192 and mental state detection subsystem 194. Action unit detector 190 is adapted to convert locations of points on the face to action units. Action unit detector 190 may be further adapted to convert motion trajectories of the points into action units. Gesture detector 192 is adapted to convert a sequence of action units to gestures. Mental state detector 194 may be adapted to infer a mental state from the action units and the gestures. As noted before, the mental states detector 194 may also be programmed, such as for example with a direct mapping function that maps the reader output directly to mental states, without detecting head and facial activity. A suitable direct mapping function enabling the mental state detector to infer mental states directly from reader output may include for example stochiastic probabilistic models such as Bayesian networks, memory based methods and other such models. The action units, gestures and mental states are stored. The action units, gestures and mental states and events may be stored continuously as a stream of data where, as a subset of the data, upon occurrence of an event the relevant action units, gestures and mental states may be tagged with the event. The stored action units, gestures or mental state are converted by the action handler 178 to an indication of a detected facial activity or mental state. Here, the action units, gestures and mental states are detected concurrently with and independent of movement of the person. In addition, sequences of action units, gestures and mental states may be stored upon occurrence of multiple events and tagged with the multiple events, where the multiple states within the sequence of mental states may occur simultaneously, overlap or occur in sequence. Action unit, detection subsystem 190 takes the data from feature point tracker 170 and buffers frames in action unit buffer 196. Detectors 198 are provided for facial features such as tongue, cheek, eyebrow, eye gaze, eyes, head, jaw, lid, lip, mouth and nose. The data from frames within action unit detection subsystem 190 is further converted to gestures in the gesture detection subsystem 192. Gesture detection subsystem 192 that buffers gestures in gesture buffer 200. Data from action units buffer 196 is fed to action units to gestures interface 202. Data from interface 202 is classified in classifiers module 204 having classifier training module 206 and classifier loading module 208. The data from frames within action unit detection subsystem 190 and from gesture detection subsystem 192 is further converted to mental states in the mental state detection subsystem 194. Mental state detection subsystem 194 takes data from gesture buffer 200 to “gestures to mental states interface” 210. Data from interface 210 is classified in classifiers module 214 having classifier training module 216 and classifier loading module 218. The training and classification allows for continuous training and classification where data may be updated in real time. Mental states are buffered in mental states buffer 212. The method of analysis described herein uses a dynamic (time-based) approach that is performed at multiple temporal granularities, for example, as depicted in FIG. 3. Drawing an analogy from the structure of speech, facial and head action units are similar to speech phonemes; these actions combine over space and time to form communicative gestures, which are similar to words; gestures combine asynchronously to communicate momentary or persistent affective and cognitive states that are analogous to phrases or sentences. A sliding window is used with a certain size and a certain sliding factor. In one embodiment, for mental state inference, a sliding window may be used, for example, that captures 2 seconds (for video recorded at 30 fps), with a sliding factor of 5 frames. Here, a task or experience is indexed at multiple levels that range from low-level descriptors of the person's activity to the person's affective or emotional tags (interest, liking, disliking, wanting, delight, frustration) cognitive tags (cognitive overload, understanding, agreement, disagreement) and memory index (e.g., whether an event is likely to be memorable or not). By way of example, a fidget index may be provided as an index of the overall face-movement at various points throughout the video. This index contributes to measuring concentration level, and may be combined also with other movement information, sensed from video or other modalities to provide an overall fidgetiness measure. In alternate embodiments, any suitable index may be combined with other suitable index to infer a given mental state.

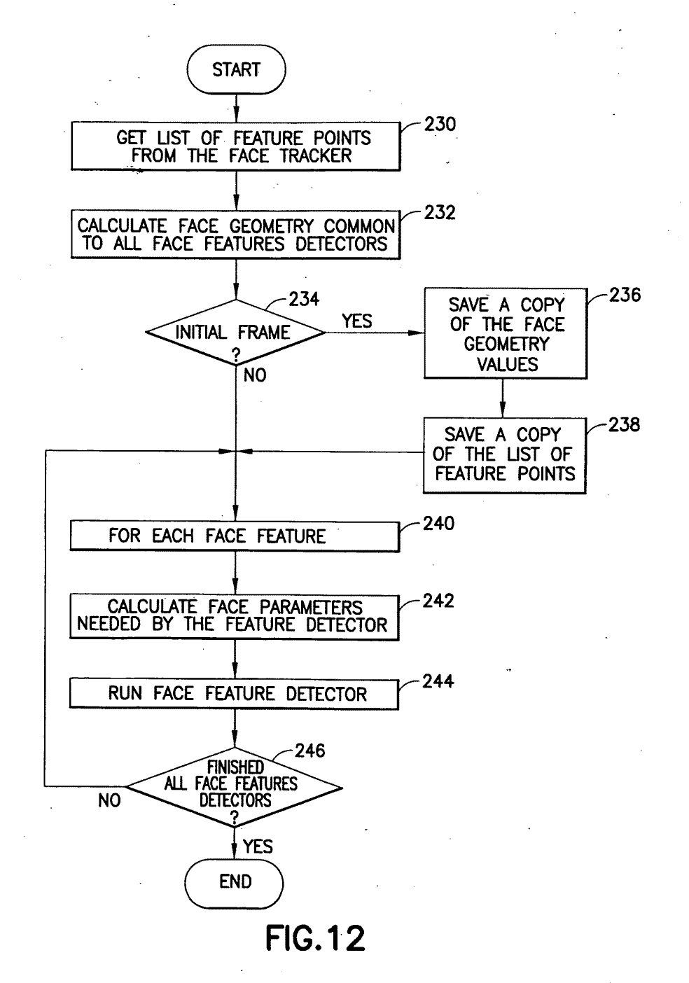

Referring now to FIG. 12, head and facial action unit analysis is shown. As described below, a list of head and facial action units that are automatically detected by the system are shown below.

| ID | Action Unit | Facial muscle |
|---|---|---|
| 1 | Inner Brow Raiser | Frontalis, pars medialis |
| 9 | Nose Wrinkler | Levator labii superioris alaquae nasi |
| 12 | Lip Corner Pull | Zygomaticus major |
| 15 | Lip Corner Depressor | Depressor anguli oris |
| 18 | Lip Puckerer | Incisivii labii superioris and Incisivii |
| | | labii inferioris |
| 20 | Lip Stretcher | Risorius w/platysma |
| 24 | Lip Pressor | Orbicularis oris |
| 25 | Lips Apart | Depressor labii inferioris |
| 26 | Jaw Drop | Masseter, relaxed Temporalis and internal |
| | | Pterygoid |
| 27 | Mouth Stretch | Pterygoids, Digastric |
| 43 | Eyes Closed | Relaxation of Levator palpebrae |
| | | superioris; Orbicularis oculi, pars |
| | | palpebralis |
| 45 | Blink | Relaxation of Levator palpebrae |
| | | superioris; Orbicularis oculi, pars |
| | | palpebralis |
| 46 | Wink | Relaxation of Levator palpebrae |
| | | superioris; Orbicularis oculi, pars |
| | | palpebralis |
| 51 | Head Turn Left | |
| 52 | Head Turn Right | |
| 53 | Head Up | |
| 54 | Head Down | |
| 55 | Head Tilt Left | |
| 56 | Head Tilt Right | |
| 57 | Head Forward | |
| 58 | Head Back | |
| 61 | Eyes Turn Left | |
| 63 | Eyes Up | |
| 64 | Eyes Down | |
| 65 | Walleye | |
| 66 | Cross-eye | |
| 71 | Head Motion Left | |
| 72 | Head Motion Right | |
| 73 | Head Motion Up | |
| 74 | Head Motion Down | |
| 75 | Head Motion Forward | |
| 76 | Head Motion Backward | |

Action units 1-58 are derived from Ekman and Friesan's Facial Action Coding System (FACS). Action unit codes 71-76 are specific to he disclosed embodiments, and are motion-based. By tracking feature points over an image sequence, a combination of descriptors are calculated for each action unit (AU). The AUs detected by the system compass both head and facial actions. Although in the disclosed embodiment, motion based action units 71-76 are shown, more or less motion based action units may be provided or derived. Here, embodiments of the methods herein include motion detection as well as texture modeling. The detection results for each AU supported by the system are accumulated onto a circular linked list; where each element in the list has a start and end frame to denote its duration. Each action is coded for a time based persistence (for example, is it a fleeting action or not) as well as intensity and speed. A maximum duration threshold is imposed for the AUs, beyond which the AU is split into a new one. Also, a minimum duration threshold is imposed to handle possibly “noisy” detections, in other words, if an AU doesn't persist for long enough it's not considered by the system. AU intensity is also computed and stored for each detected AU. Examples of head AUs that may be detected by the system may include the pitch actions AU53 (up) and AU54 (down), yaw actions AU51 (turn-left) and AU52 (turn-right), and head roll actions AU55 (tilt-left) and AU56 (tilt-right). The rotation along the pitch, yaw and roll may be calculated from expression invariant points. These points may include the nose tip, nose root and inner and outer eye corners. For instance, yaw rotation may be computed as the ratio of the left to right eye widths, while roll rotation may be computed as the rotation of the line connecting the inner eye corners. FACS head AUs are pose descriptors. By way of example, AU53 may depict that a head is facing upward, regardless of whether it is moving or not. Similarly, motion and geometry-based AU detection may be provided in order to be able to detect movement and not just pose, for example action units AU71-AU76. The lip action units (lip corner pull AU12, lip stretcher AU16, lip depressor AU18, lip puckerer AU19) may be computed through the lip corners, mouth corners, eye corners feature points and the head scale where the latter may be used to normalize against changes in pose due to head motion towards or away from the camera. On an initial frame, the difference in distance between the mouth center and the line connecting the 2 mouth corners may be computed. Second, the distance between the average distance between the mouth corners and the distance calculated in the initial video frame may also be computed. At every frame, the same parameters are computed and the difference indicated the phase and magnitude of the motion, which may be used to depict the specific lip AU. To compute the mouth action units (lips part AU25, mouth stretch AU26, jaw drop AU27), the feature points related to the nose (nose root and nose tip) and the mouth (Upper Lip Center, Lower Lip Center, Right Upper Lip, Right Lower Lip, Left Upper Lip, Left Lower Lip) may be used. Like the lip action units, the mouth action units may be computed using mouth parameters during the initial frame compared to mouth parameters at the current frame. For example, at the initial frame, a ratio is computed of: 1. the distance of the line connecting the nose root and the upper lip center, 2. the average of the lines connecting the upper and lower lip centers, and 3. the distance of the line connecting the nose tip and the lower lip centers. The same ratio is computed at every frame. The difference between the ratio calculated at the initial frame and the one calculated in the current frame is threshholded to detect one of the mouth AUs and the respective intensity. To compute the eyebrow action units (AU 1+2), the eyebrow inner, center and outer points may be detected, as well as the eye inner, center and outer points. Calculate distance between them, and account for head motion. If it exceeds a certain threshold then it is considered an AU1+2. The algorithm in FIG. 12 retrieves 230 the list of feature points from the face tracker and calculates 232 face geometry common to all face features detectors. If it is on the initial frame 234, a copy 236 of the face geometry values is saved and a copy 238 of the list of feature points is saved. If it is not on the initial frame 234, for each face feature 240, face parameters 242 needed by the feature detector are calculated and the face feature detector 244 is run until all face feature detectors 246 are finished.

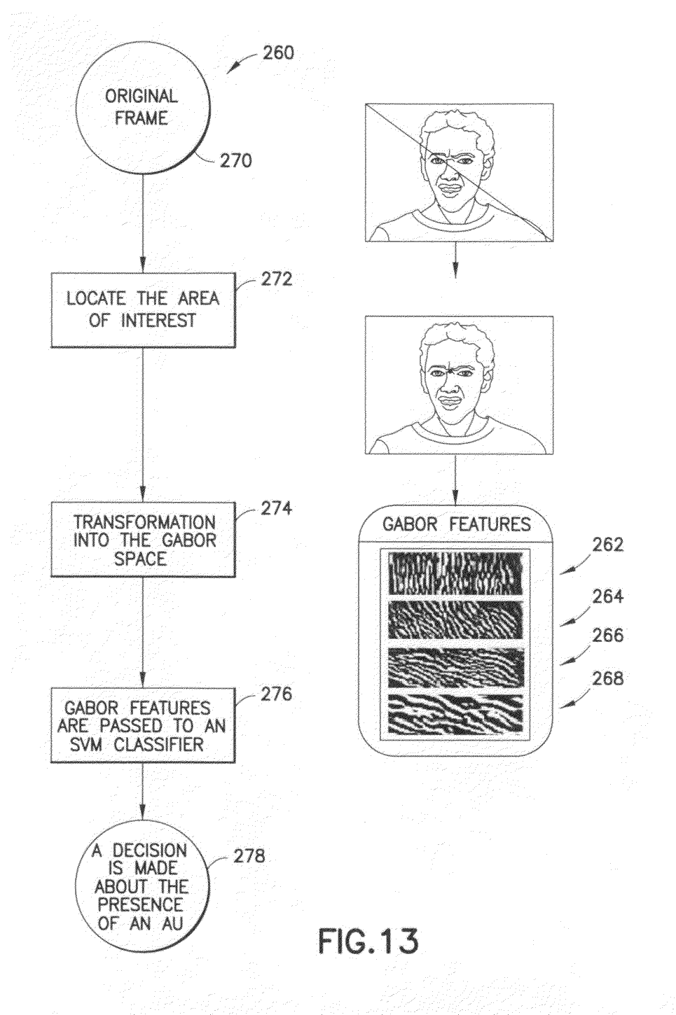

Referring now to FIG. 13, a schematic diagram graphically depicts texture based action unit analysis 260 using, for example, Gabor jets around areas of interest in the face. As can be seen in FIG. 13, the feature points define a bounding box 262, 264, 266, 268 around a certain facial area. For the texture-based AUs, fiducial landmarks are used to define a region of interest centered around or defined by these points, and it is the texture of this region is of interest. Analysis of the texture or color patterns and changes within this bounded area are also used to identify various AUs. In the disclosed embodiment this method may be used to identify the nose wrinkle AU (AU 9 and 10) as well as eye closed (AU 43), eye blink and wink, eyebrow furrowing (AU 4). In alternate embodiments, more or less AU's may be detected by this method. This method uses Gabor jets to describe textured facial regions, which are then classified into AUs of interest. The analysis 260 takes, block 270, an original frame, locates 272 an area of interest, transforms 274 the area of interest into the gabor space, passes 276 the gabor features to a Support Vector Machine (SVM)—classifier and makes a decision 278 about the presence of an action unit. Gabor jets describe the local image contrast around a given pixel in angular and radial directions. Gabor jets are characterized by the radius of the ring around which the Gabor computation will be applied. Gabor filtering involves convolving the image with a Gaussian function multiplied by a sinusoidal function. The Gabor filters function as orientation and scale tunable edge detectors. The statistics of these features can be used to characterize underlying texture information. The Gabor function is defined as:

g(t)=ke{i\theta}w(at)s(t)

where w(t) is a Gaussian function and s(t) is a sinusoidal function. For each action unit of interest, a region of interest is defined, and the center of that region is computed and used as the center of the Gabor jet filter for that action unit. For instance, the nose top defines a region of interest for the nose wrinkle region with a pre-defined radius, while the center of the pupil defines the region of interest for deciding whether the eye is open or closed. Different sizes for the regions of interest may be used. This region is extracted on every frame of the video. The extracted image is then passed to the Gabor filters with 4 scales and 6 orientations to generate the features. This method allows for action unit detection that is robust to head rotation, in real-time. Also, this approach makes it possible to train new action units of interest provided that there are training examples and that it is possible to localize the region of interest. In the embodiment shown, feature points are detected and used as an anchor to speed shape and texture detection. In the embodiment shown, texture based action unit analysis may be used to identify both static and motion based action units.

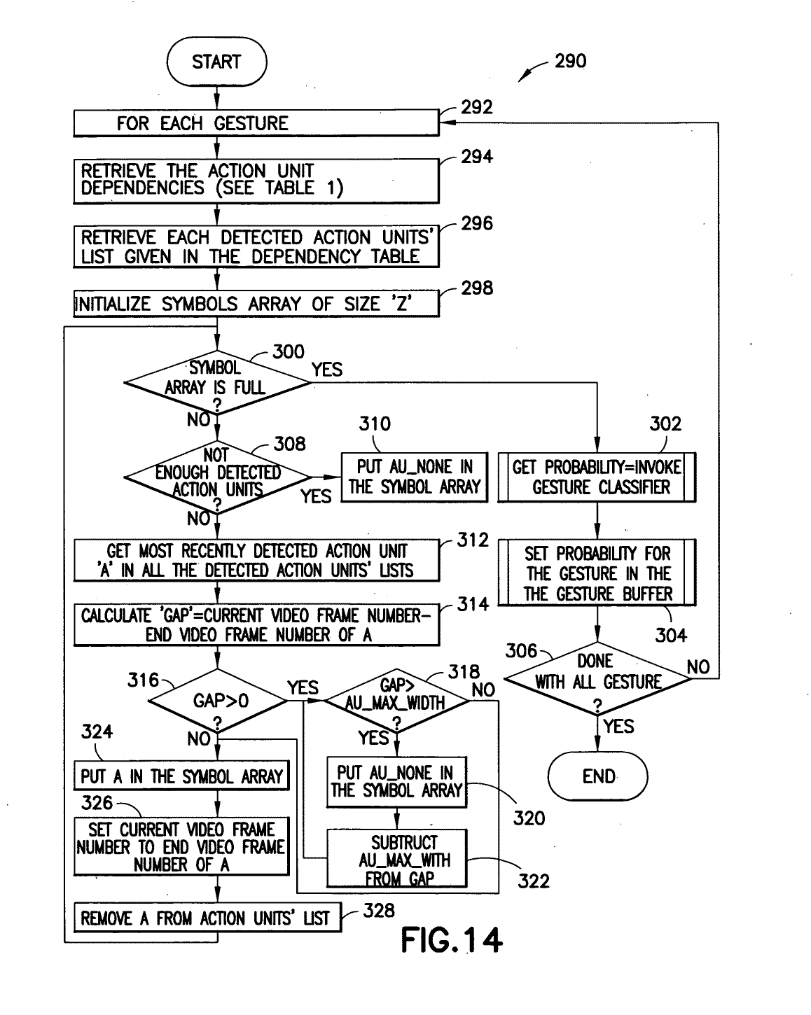

Referring now to FIG. 14, there is shown a flow chart graphically illustrating head and face gesture classification 290 in accordance with an exemplary embodiment. The FIG. 14 flowchart shows an exemplary process that may be used in compiling an array of the most recent AU'-s. For each gesture, block 292, action unit dependencies block 294 are retrieved as seen in the exemplary dependencies table below. Each detected action units' list block 296 given in the dependency table is then retrieved and symbols array of size “Z”, block 298, is then initialized. If the symbol array is full, the probability=invoke gesture classifier block 302 is gotten and the probability for the gesture in the gesture buffer block 304 is set. If all gestures are not done block 306, the algorithm goes back to the start. If the symbol array is not full, block 300, and there are not enough detected action units, block 308, AU_NONE is put block 310 in the symbol array. If there are enough action units detected block 308, then the most recently detected action unit “A” in all the detected action units lists block 312 is retrieved and GAP=current video frame number−end video frame number of A block 314 is calculated. If in block 316 the GAP>0 and the GAP>AU_MAX_WIDTH block 318 then AU_NONE is put in the symbol array block 320 and AU_MAX_WIDTH is subtracted block 322 from GAP. If GAP is not >0 then A is put block 324 in the symbol array, the current video frame number is set block 326 to end video frame number of A and A is removed block 328 from the action units' list. To infer the social signals or communicative nature of head and facial AUs, it is necessary to consider a sequence of AUs over time. For instance, a series of head up and down pitch movements may signal a head nod gesture. Thus, each gesture is associated with one or more AUs, which we refer to as the gesture's AU dependency list. By way of example, the table below lists the gestures that a disclosed embodiment may include as well as associated AUs that the gesture depends on. An exemplary list of gestures and their AU dependencies are summarized in the table below.

TABLE
List of Gestures and their action unit dependencies

| Gesture_ID | Gesture_Description | Dependency_1 | Depedency_2 |
|---|---|---|---|
| 501 | HeadNod | Head move up | Head move down |
| 502 | HeadShake | Head motion left | Head motion right |
| 505 | PersistentHeadTurnRight | Head turn left | Head turn right |
| | | (AU51) | (AU52) |
| 506 | PersistentHeadTurnLeft | Head turn left | Head turn right |
| | | (AU51) | (AU52) |
| 507 | PersistentHeadTiltRight | Head tilt left | Head tilt right |
| | | (AU55) | (AU56) |
| 508 | PersistentHeadTiltLeft | Head tilt left | Head tilt right |
| | | (AU55) | (AU56) |
| 509 | HeadForward | Head motion | Head motion |
| | | forward | backward |
| 510 | HeadBackward | Head motion | Head motion |
| | | forward | backward |
| 511 | Smile | Lip Corner Pull | Lip puckerer (AU18) |
| | | (AU12) | |
| 514 | Stretch | Lip Stretcher | Lip Puckerer (AU18) |
| | | (AU20) | |
| 512 | Puckerer | Lip Corner Pull | Lip puckerer (AU18) |
| | | (AU12) | |
| 513 | EyeBrowRaise | Inner Brow Raiser | Outer Brow Raiser |
| | | (AD1) | (AU2) |

By way of example, a head nod has a dependency on head_up and head_down actions. In addition, AU_NONE may be defined to represent the absence of any detected AUs. Each gesture is represented as a probabilistic classifier encoding the relationship between the AUs and gestures. The approach to train each classifier is supervised, meaning examples depicting the relationship between AUs and a gesture are needed. To run the classifier for classification, a sequence of the most recent history of relevant AUs per gesture needs to be compiled. The algorithm to compile a sequence of the most recent history of relevant AUs per gesture is shown in FIG. 14. For each gesture, the list of all its AU dependencies is retrieved 294, and the corresponding AU lists are loaded. The lists are parsed to get the most recent AU, defined as the AU that ended the most recently. If the time elapsed between the current time and most recent AU exceeds a specified threshold, the action unit depicting a neutral facial movement is included. The algorithm to get the most recent AU is repeated, moving backward in history until enough AUs are identified per gesture. When a sequence of most recent AUs is compiled for each gesture, the vector is input to the classifier 502 for inference, yielding a probability for each gesture. Gesture classifiers are independent of each other and can co-occur.

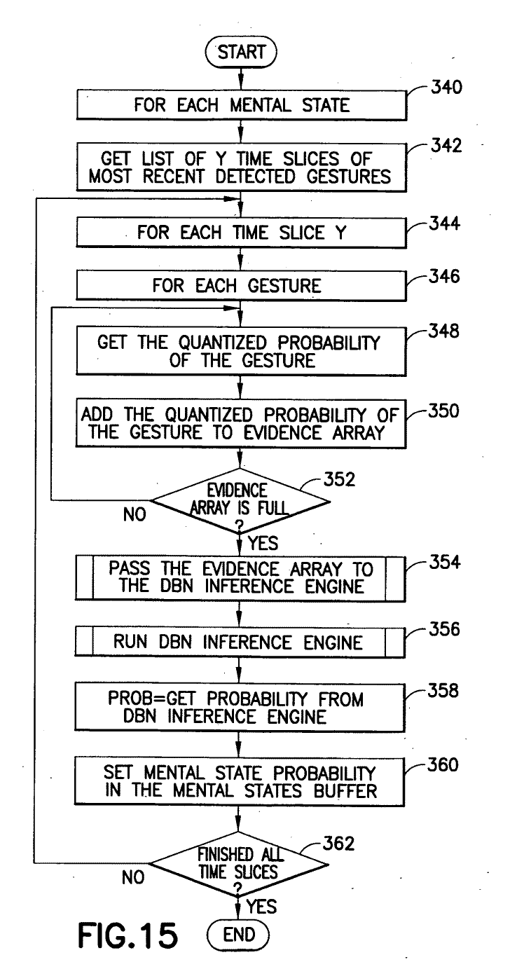

Referring now to FIG. 15, mental state classification is shown as a gesture to mental state recognition flowchart. For each mental state block 340, a list of Y time slices is retrieved block 342 of the most recent detected gestures. For each time slice Y block 344 and each gesture block 346 the quantized probability of the gesture is retrieved block 348 and the quantized probability of the gesture is added, block 350 to the evidence array. If the evidence array is full block 352, the evidence array is passed block 354 to the DBN inference engine and PROB=Get Probability from DBN inference engine block 358 and the mental state probability is set block 360 in the mental state buffer. If all time slices are not finished, the algorithm goes back to block 344 for each time slice Y. The identified head and facial gestures are used to infer a set of possible momentary affective or cognitive states of the user. These states may include, for example, interest, boredom, excitement, surprise, delight, frustration, confusion, concentration, thinking, distraction, listening, comprehending, nervous, anxious, worried, bothered, angry, liking, disliking, curiosity or otherwise. Mental states are represented as probabilistic classifiers that encode the dependency between specific gestures and mental states. The current embodiment uses Dynamic Bayesian Networks (DBN's) as well as the simpler graphical models known as Hidden Markov Models (HMM's) but the invention is not limited to these specific models. However, models that capture dynamic information are preferable to those that ignore dynamics. Each mental state is represented as a classifier. Thus, mental states are not mutually exclusive. The disclosed embodiments allow for simultaneous states to be present having different probabilities of occurrence, or levels of confidence in their recognition. Thus, the disclosed method represents the complex relationship between mapping from gestures to mental states. Optionally, a feature selection method may be used to select the gestures most important to the inference of a mental state. To train a mental state, an input sequence of gestures representative of that mental state is needed. This is called the evidence array. Evidence arrays are needed for positive as well as negative examples of a mental state. A mental state evidence array may be represented, for example as a list of 1's or 0's representing each detected \ not detected gesture defined in the system. Each cell in the array represents a defined gesture, 1 is an indication that this gesture was detected, whereas 0 is an indication that it was not. For example, if number of gestures defined in the system are 12, the array would consist of 12 cells. The gestures are classified into mental states where for each time slice, for each gesture, the probabilities of each gesture are quantized to a binary value to be compiled as input to the discrete dynamic Bayesian network. The gestures are compiled over the course of a specified sliding window. The computational model can predict the onset of states, e.g., confusion, and could thus alert a system to take steps to respond appropriately. For example, a system might offer another explanation if it detects sustained confusion. Valence Index consists of Patterns of action units and head movement over an established window of time are automatically labeled with a likelihood that they correspond to positive or negative facial-head expression. The disclosed embodiments include a method to compute the Memorable Index, which is computed as a weighted combination of the uniqueness of the event, the consequences (for instance, you press cancel by mistake and all the data you entered over the last half-hour is lost), the emotion expressed, its valence and the intensity of the reaction. This is calculated over the course of the video as well as at certain key points of interest (e.g., when data is submitted or towards the end of an interaction). A Memorable Index is particularly important in learning environments to quantify a student's experience and compare between different approaches to learning, or in usability test environments, to help identify problems that the designers probably should fix. It also has importance in applications such as online shopping or services for identifying which options provide better sales and service experiences.

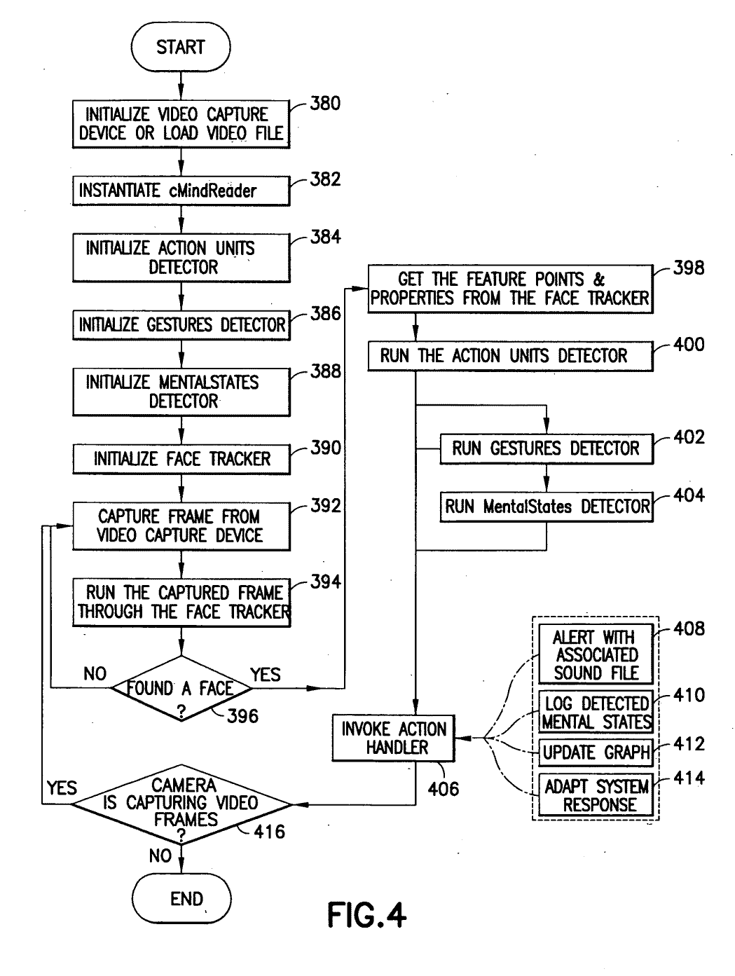

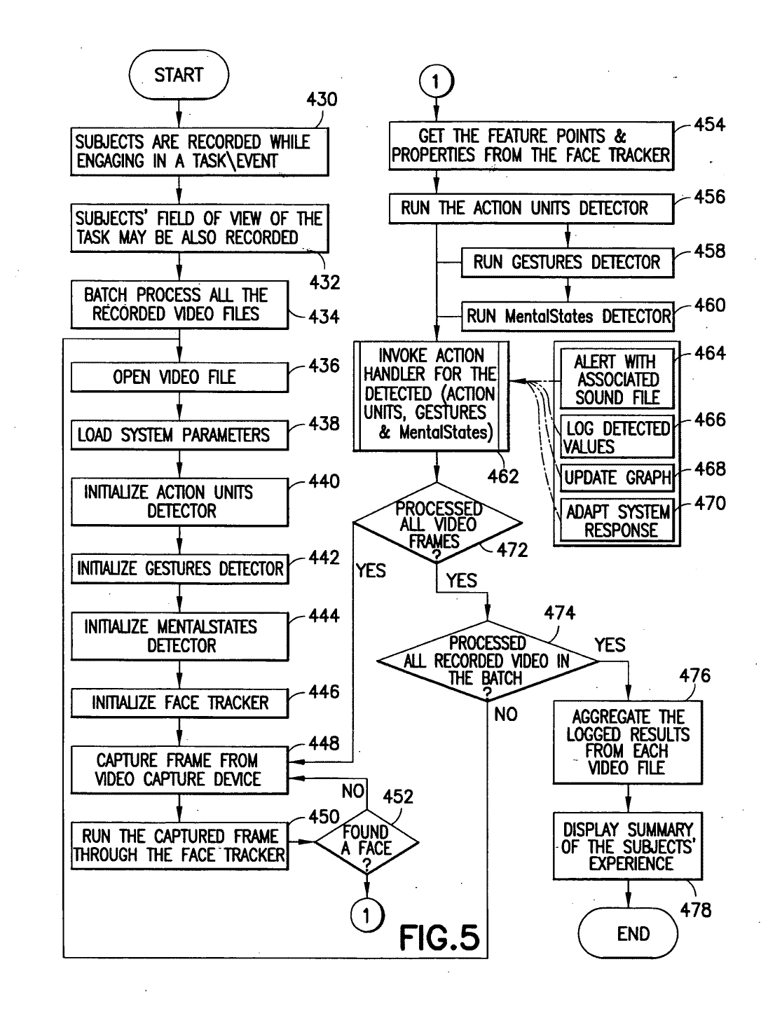

Referring now to FIG. 4, a flow chart illustrating an automatic real-time analysis is shown. Here, FIG. 4 shows a method for the automatic, real-time analysis of head and facial activity and the inference, tagging, and prediction of people's affective and cognitive experiences, and for the real-time decision-making and adaptation of a system to a person's state. The algorithm of FIG. 4 begins by initializing video capture device or loads a video file 380, cMindReader 382, action units detector 384, gesture detector 386, mental states detector 388 and face tracker 390. Frames are captured 392 from the video capture device 392 and captured frames are run 394 through the face tracker. If a face is found 396 then the feature points and properties from the face tracker are retrieved 398 and action units detector 400, gestures detector 402 and mental state detector 404 are run. Action handler 406 is then invoked with corresponding actions, such as alerting 408 with an associated sound file, logging a detected mental state 410, updating a graph 412 or adapting a system response 414. If the camera continues to capture frames 416, the algorithm continues to capture 392 and process the frames. FIG. 4 details the algorithm for the automatic, real-time analysis. One or more persons, each engaged with a task are facing a video camera. A frame gabber module grabs the frames at camera speed, which is then passed to the system for analysis. The parameters and classifier are initialized. A face-finder module is invoked to locate a face within the frame. If a face is found, a facial feature tracker then locates a number of facial landmarks on the face. The facial landmarks are used in the geometric and texture-based action unit recognition. Optionally, the results may be logged, plotted, or may invoke some form of auditory, visual or tactile feedback as previously described. The action units are compiled as evidence for gesture recognition. Optionally, the results may be logged, plotted, or may invoke some form of auditory, visual or tactile feedback as previously described. The gestures over a certain period of time are compiled as evidence for affective and cognitive mental state recognition. Optionally, the results may be logged, plotted, or may invoke some form of auditory, visual or tactile feedback. The results of the analysis can be fedback to the system in real-time to adapt the course of the task, or response given by a system. The system could also be linked to a reward or point system. In the real-time mode, the apparatus can have a wearable, portable form-factor and wearers can exchange information about the affective and cognitive states. Examples of automatic real-time analysis involves customer research, product usability and evaluation, advertising: customers are asked to try out a new product (which could be a new gadget, a new toy, a new beverage or food, a new automobile dashboard, a new software tool, etc) and a small camera is positioned to capture their facial-head movements during the interactive experience. The apparatus yields tags that describe liking and disliking, confusion, or other states of interest for inferring where the product use experience could be improved. A researcher can be visualizing these results in real-time during the customer's interaction. Another application may be where the system is used as a conversational guidance systems and intervention for autism spectrum disorders where the system performs automatic, real-time analysis, inference, tagging of facial information which is presented in real-time as graphs as well as other output information beyond graphs—e.g. summarizing features of interest (such as frowns or nose wrinkles) as bar graphs that can be visually compared to neutral or positive features (such as eyebrow raises or smiles involving only the zygomate). The output can also be mapped to LED, sound or vibration feedback. Another application involves an intelligent tutoring system, driver monitoring system, live exhibition where the system adapts its behavior and responses to the person's facial expressions and the underlying state of the person.

Below, an algorithm for sequence of facial and head movement and analysis is shown. For descriptive purposes only, the algorithm may be considered as having generally four sequences: 1) Initialization & Facial feature tracking: 2) AU-level, head and facial action unit recognition, 3) Gesture-level: Head motion and facial gestures recognition and 4) Mental State-level: mental state inference. In alternate embodiments, the algorithm may be structured or organized in any desired number of sequences. As may be realized the below listed algorithm is graphically illustrated generally in FIG. 4. Initialization & Facial feature tracking comprises Initializing video capture device(s) or load video file(s) and Initiating and initializing the detectors (see also FIG. 2). The detectors, as noted before include an Action Units Detector where the detector's data structures are initialized. The detectors further include a Gestures Detector where the process initializes the detector's data structures and trains or loads the display HMMs. The detectors further include a Mental States Detector where the process initializes the detector's data structures and learns DBN model parameters and select best model structure. In accordance with the algorithm the face tracker is initialized to find the face. In the exemplary embodiment it is provided to track facial feature points. AU-level: head and facial action unit recognition comprises a Function to detect Action Units( ) which has components including 1) Derive motion, share and color models of facial components and head, 2) Head pose estimation->Extracting head action units and 3) Storing the output in the Action Units Buffer. The algorithm further comprises appending the Action Unit Buffer to a file. Gesture-level: Head motion and facial gestures recognition comprises a Function to detect Gestures( ) which has components 1) Infer the action units detected in the predefined history time frame, 2) Input the action units to the display HMMs, 3) Quantize the output to binary and 4) Store both the output percentages and the Quantized output in the Gestures Buffer. to the algorithm further comprises appending the Quantized Gesture Buffer to a file. The Mental State-level: mental state inference comprises a Function to detectMentalStates( ) which has components 1) Infer the Gestures detected in the predefined history time frame, 2) Construct observation vector by concatenating s outputs of display HMM, 3) Input observations as evidence to DBN inference engines and 4) Store both the output percentages and the Quantized output in the Mental States Buffer. The Quantized Mental States may also be appended to a file. The algorithm is set forth below:

Algorithm 1: Sequence of Facial and Head Movement Analysis.

### Initialization & Facial Feature Tracking:

Initialize video capture device or load video file
Instantiate and initialize the detectors
   Action Units Detector
      Initialize the detector's data structures
   Gestures Detector
      Initialize the detector's data structures
      Train or Load display HMMs
   Mental States Detector
      Initialize the detector's data structures
      Learn DBN model parameters and select best model structure
Initialize face tracker
Find the face
Track facial feature points

### AU-Level: Head and Facial Action Unit Recognition

Function detectActionUnits( )
   Derive motion, share and color models of facial components and head
   Head pose estimation->Extract head action units
   Store the output in the Action Units Buffer
Append the Action Unit Buffer to a file

### Gesture-Level: Head Motion and Facial Gestures Recognition

Function detectGestures( )
   Infer the action units detected in the predefined history time frame.
   Input the action units to the display HMMs
   Quantize the output to binary
   Store both the output percentages and the Quantized output in the Gestures Buffer
Append the Quantized Gesture Buffer to a file

### Mental State-Level: Mental State Inference

Function detectMentalStates( )
   Infer the Gestures detected in the predefined history time frame.
   Construct observation vector by concatenating s outputs of display HMM
   Input observations as evidence to DBN inference engines
   Store both the output percentages and the Quantized output in the Mental States Buffer
Append the Quantized MentalStates to a file

Referring now to FIG. 5, there is shown automatic offline analysis. The algorithm of FIG. 5 begins where subjects are recorded 430 while engaging in a task or event and where the subjects field of view may also be recorded 432. All of the recorded video files are then recorded 434 and the video file opened 436. System parameters are then loaded 438 and the action units detector 440, gesture detector 442, mental states detector 444 and face tracker 446 are initialized. Frames are captured 448 from the video capture device and captured frames are run 450 through the face tracker. If a face is found 452 then the feature points and properties from the face tracker are retrieved 454 and action units detector 456, gestures detector 458 and mental state detector 460 are run. Action handler 462 is then invoked with corresponding actions, such as alerting 464 with an associated sound file, logging a detected mental state 466, updating a graph 468 or adapting a system response 470. If all video frames are not processed 472, the algorithm continues to capture 448 and process the frames. If all video frames are processed 472, and all recorded video in the batch are processed 474, the logged results from each video file are aggregated 476 and a summary of the subjects' experience are displayed 478. Here, FIG. 5 illustrates a method for the 1) automatic, offline analysis of head and facial activity and the inference, tagging, and prediction of people's affective and cognitive experiences, 2) aggregation of results across one or more persons, and 3) synchronization with the event video and/or log data to yield insight into a person's affective or cognitive experience. One or more persons are invited to engage in a task while being recorded on camera. The person's field of view or task may also be recorded. Once the task is completed, recording is stopped. The resulting video file or files are then loaded into the system for analysis. Alternatively, the system herein can analyze facial videos in real-time without any manual or human processing or intervention as has been previously described. For a video (an image sequence), one frame is automatically extracted at a time (at recording speed). The parameters and classifier are initialized. A face-finder module is invoked to locate a face within the frame. If a face is found, a facial feature tracker then locates a number of facial landmarks on the face. The facial landmarks are used in the geometric and texture-based action unit recognition. Optionally, the results may be logged, plotted, or may invoke some form of auditory, visual or tactile feedback. The action units are compiled as evidence for gesture recognition. Optionally, the results may be logged, plotted, or may invoke some form of auditory, visual or tactile feedback. The gestures over a certain period of time are compiled as evidence for affective and cognitive mental state recognition. Optionally, the results may be logged, plotted, or may invoke some form of auditory, visual or tactile feedback. This analysis yields a meta-analysis of the person's state: the temporal progression and persistence of states over an extended period of time, such as the course of a trial. Once all the videos have been processed, the results are synchronized with the event video and/or data logs. The disclosed embodiments include a method for aggregating the data of one person over multiple, similar trials (for instance, watching the same advertisement, or filling in the same tax form several times, or visiting the same web site multiple times). The disclosed embodiments also include a method for time-warping and synchronizing facial (and other data) events. The disclosed embodiments also include a method for aggregating the data across multiple people (for instance, if multiple people were to view the same advertisement). The final results would indicate general states such as customer delight in usability or experience studies, or liking and disliking in consumer beverage or food taste-studies, or level engagement with a robot or agent. The aggregation is useful in customer research, product usability and evaluation, advertising, where typically many customers are asked to try out a new product (which could be a new gadget, a new toy, a new beverage or food, a new automobile dashboard, a new software tool, etc) and a small camera is positioned to capture their facial-head movements during the interactive experience. The apparatus yields tags that describe liking and disliking, confusion, or other states of interest for inferring where the product use experience could be improved. This would typically be done after the customers are done with the interaction. For scenarios where multiple persons are taking the same task or going through the same experience, it is desirable to be able to perform aggregate analysis such as to aggregate data from these multiple persons. There are two scenarios to consider here. In the first case, the events are aligned across all participants (e.g., all participants watching same advertisement or trailer, so facial expressions are lined up in time across all participants), the aggregate function may be a simple sum or average function that counts number of occurrences of certain states of interest at specific event markers or time stamps. In the second case, the events are not exactly lined up in time (e.g., in a beverage tasting study where people can take varying times to taste the beverage and answer questions). In that case, counts of facial and heading movements (and/or gestures and mental state information) is aggregated per event of interest, which is defined as a period of time during which an event occurs (e.g., within the first 10 seconds after a sipping event occurs in the beverage tasting scenario). The output can also be aligned across stratified groups of participants, e.g., all females vs. males; all Asian vs. Hispanics.

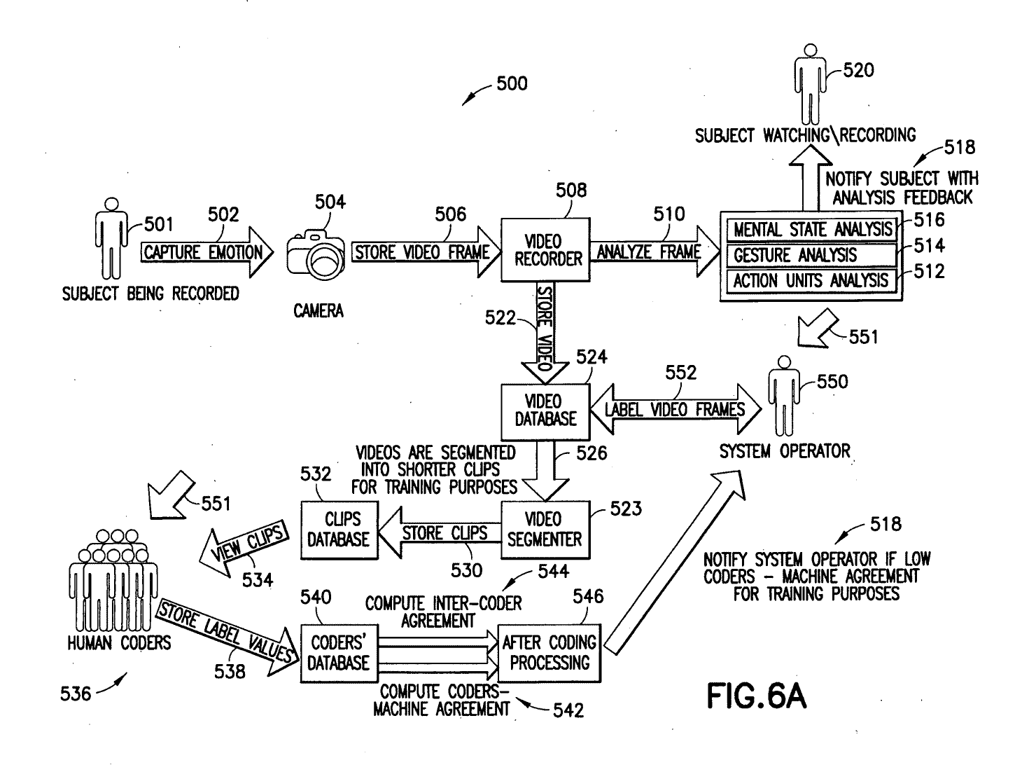

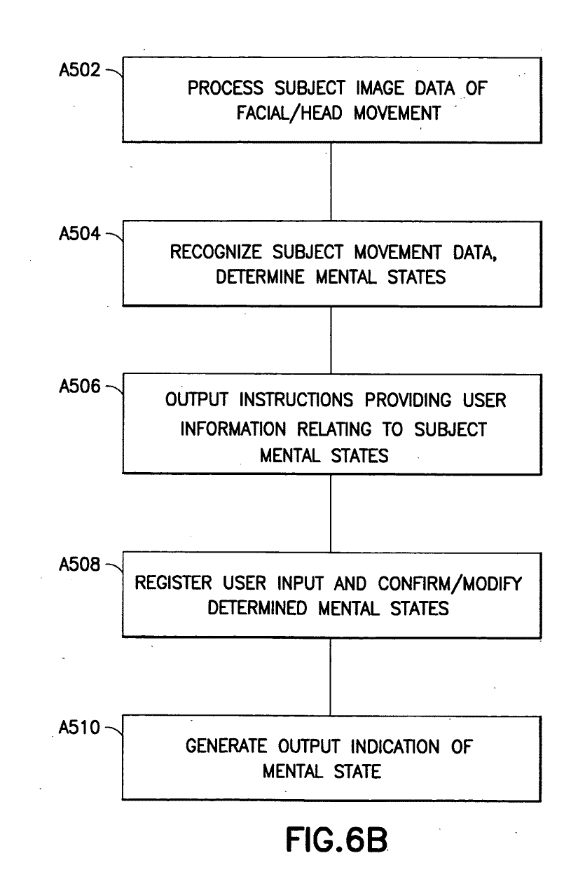

Referring now to FIG. 6a-6b, there are shown exemplary assisted analysis system 500, 500′ and process in accordance with other exemplary embodiments. As will be described further unlike conventional systems below, the analysis mode wherein the system provides information to a user and accepts input from the user may be performed substantially in real time or may be offline. A first exemplary embodiment of a system 500 and process for facial and head activity and mental state analysis is shown in FIG. 6a. The system shown in FIG. 6a may perform the analysis of facial/head activity and mental state, including human observer/coder interface or input, in substantially real time. For example a human observer 536 is tagging in real-time while being assisted by the machine 512, 514, 516. In the exemplary embodiment, the system may include some display, or other user readable indictor, providing the user/observer with information regarding the event, the person's actions in the event, as well as processor inferred head and facial activity information, mental state information and so on. For example, the observer 550 watches a person's face on display 501 and from information thereon may identify events, AU's, gestures and mental states and tag the events in real-time while in parallel, the system tells (via a suitable indicator) 551 the observer 536, also in real-time the action or gesture, for example, “look observer this is a smile”. The observer 536 may then using an appropriate interface 538 tag a corresponding event with the smile or not, depending on the observer's 536 personal judgement of the system's help and what the observer is seeing. As may be realized, the input interface 538 may be communicably connected to the system interface 172 (see FIG. 2) and hence to one or more of the action unit detector 190, the gestures detector 192, the mental states detector 194 and action handler 178. Here, action units, gestures and mental states are analyzed in a an assisted analysis where the semi-automatic analysis comprises a real time analysis of the facial activity and mental state, and real time tagging of the mental state by the human observer.

Another exemplary embodiment of an assisted analysis system similar to system 500 and process for facial and head activity and mental state analysis is illustrated in FIG. 6b. FIG. 6b is a block diagram graphically illustrating a system, for example similar to assisted system 500 of the exemplary embodiment shown in FIG. 6a, and exemplary process that may be effected thereby. The arrangement and order shown in FIG. 6b is exemplary and in alternate embodiments the system and process sections may be arranged in any desired order. In the exemplary embodiment, in block A502, the assisted or semi-automatic system, such as system 500 (see also FIG. 6a) may process image data indicative of facial and head movements (e.g. taken with camera 504) of the subject (e.g. subject 501) to recognize at least one of the subject's movements and, in block A504 may determine at least one mental state of the subject (e.g. with modules 512-516) from the image data. As may be realized and is described further herein, the processing of the data and determination of the mental state(s) may comprise calculating (e.g. with modules 512-516) a value indicative of certainty or of a range of certainties or probability or a range of probabilities regarding the mental state. In block A506, the system may output instructions for providing to one or more human coders (e.g. via image or clips data 524-534 to coders 551) information relating to the determined mental state(s). As is described further herein, the instructions to the human coder(s) may comprise substantially real time information regarding the user's mental state(s). In block A508, the system further process data reflective of input from the human coders, and based at least in part on the registered input, confirming or modifying said determination of the mental state(s). In block A510, the system may generate, with a transducer or other suitable device an output of humanly perceptible stimuli (e.g. indicator 551, see also FIG. 6a) indicative of the mental state(s). Thus, the system shown in FIG. 6b may perform the analysis of facial/head activity and mental state with the human observer/coder interface or input to the system and analytic process being substantially real time or offline (e.g. after the occurrence of the event, the human observer/coder using previously recorded video or other data).

In addition to the operation of systems 500, described above and with respect to FIGS. 6a and 6b, systems 500, may also operate as described below. For subject 501, being recorded emotions 502, may be captured with camera 504, and video frames stored 506, with video recorder 508. As described, frames may be analyzed 510 via action unit analysis 512, gesture analysis 514, or mental state analysis 516. The subject may be notified 518, with analysis feedback with the subject watching and/or recording 520. The video may be stored 522, in video database 524, and segmented into shorter clips 526, according to their labels to a video segmenter 528. The stored clips 530, may be maintained in clips database 532, with the video clips accessed by human coders 536, where coders 536, store 538, label values to a coders' database 540. Intercoder agreement 544, and coders—machine agreement 542 may be computed after coding processing 546, and system operator 550 is notified 548 of low coders—machine agreement for training purposes where operator 550 labels the video frames 552. Here, there is shown a method for the semi-automatic, real-time analysis of video, combining real-time analysis and visualization of a person's state with real-time labeling of a person's state by a human observer. The system and matter described herein allow for the identification of affective and cognitive states during dynamic social interactions. The system analyzes real-time video feeds and using computer vision to ascertain facial expression. By analyzing the video feed to discern what emotions are currently being exhibited, the system can illustrate on the screen which facial gestures (e.g. a head nod) are being observed, which can allow for more accurate assisted tagging of emotions (for example, agreeing or otherwise). The system allows for both real-time emotion tagging and offline tagging. Videos recorded by the system are labeled in real-time by the person operating the system. The real-time labels are used as a segmenting guide later, with each video segment constructed as a certain length of video recorded before and after a real-time tag. Later, labelers watch the recorded videos, without knowledge of the original tag, and each labeler applies their own tag to the videos from a set of tags including the original tag and some foils. The labels applied by each labeler for a given video are then collected and analyzed. Inter-coder Agreement is calculated by inferring what percentage of offline labelers provided the same label to the video as the person created and labeled it in real-time. Alternatively, inter-coder agreement is inferred by taking the number of labels given most often to a given video as a fraction of the total number of labels for the video.

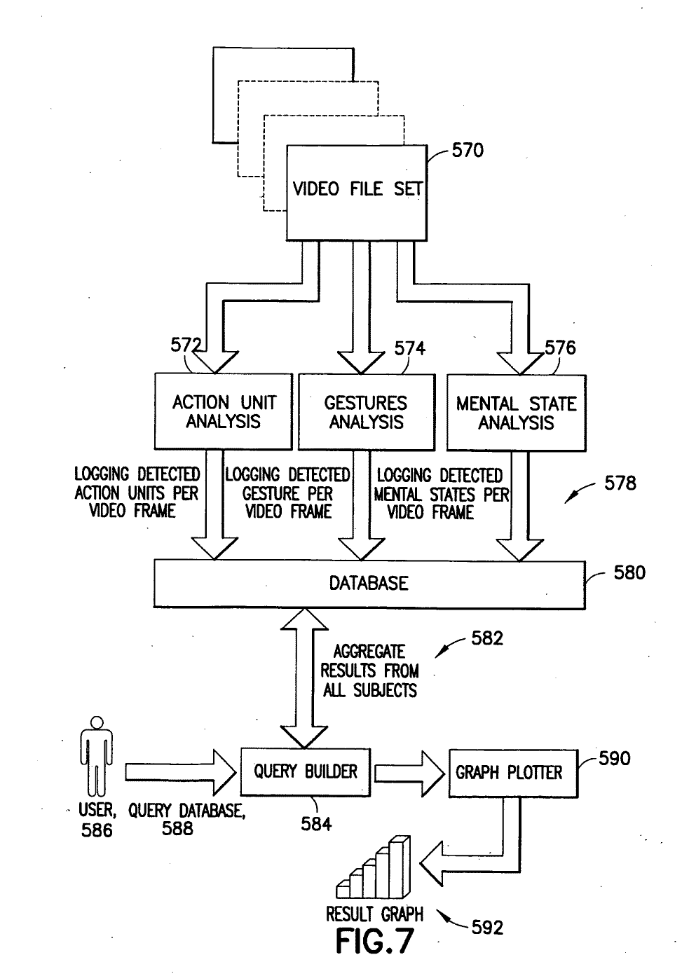

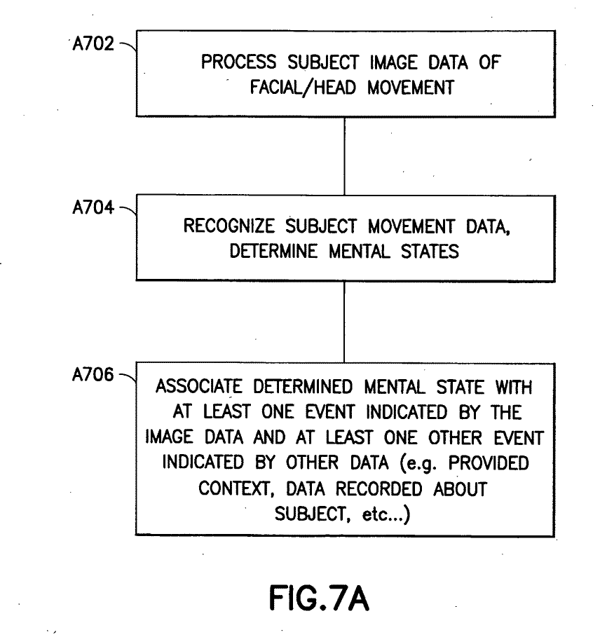

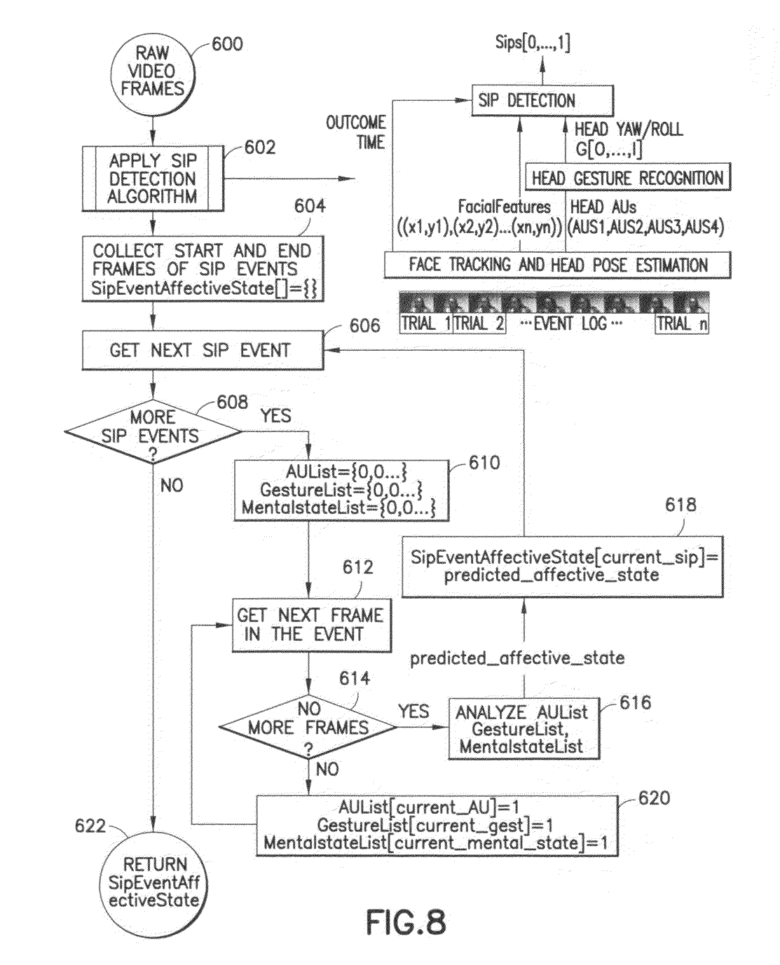

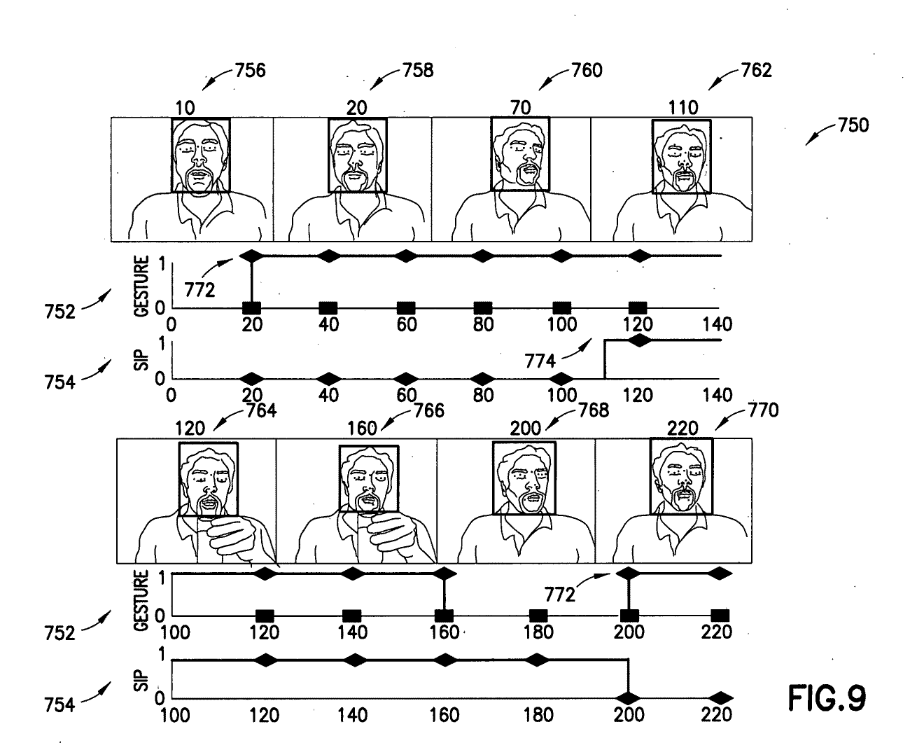

Referring now to FIGS. 7 and 8, semi-automatic offline analysis is shown. Here, FIG. 7 shows a method for the semi-automatic, offline analysis of video, combining offline analysis of videos with one or more human coders, as well as between machine and coders is computed. Videos with low inter-coder reliability are flagged for system operator. Video file set 570 is processed with action unit 572, gesture 574 and mental state 576 analysis. Detected 578 action units, gestures and mental states per frame are stored in database 580 and results 582 are aggregated from all subjects to query builder 584. Further, an event recorder correlates one or more events to one or more states. Conversely, one or more states may be correlated by the system to more than one event. This is graphically illustrated in the block diagram shown FIG. 7a. The order and arrangement shown in block diagram of FIG. 7a is representative and in alternate embodiments the system may have any other desired arrangement and order. In the exemplary embodiment, the assisted or semi-automatic system, such as system 500 (see also FIG. 6a), may process, such as in a manner similar to that described previously, image data indicative of facial and head movements of the subject to recognize at least one of the subject's movements in block A702, and in block A704 may determine at least one mental state(s) of the subject from the image data. In the exemplary embodiment, the system may, in block A706, associate determined mental state(s) with at least one event indicated by the image data and at least one other event indicated by a data set different than the image data, such as for example content of material addressed by the subject, data recorded about the subject, or other such data. User 586 may query 588 the database and output results, for example to a graph plotter 590 and resulting graph 592. Here, FIG. 9 shows detecting events of interest, for example, sipping a beverage. Although FIG. 9 is used in the context of a sip, other applications may be applied, for example, other interactions or events and senses such as reading on a screen or eye movement may be provided. Sip detection algorithm 602 is applied to raw video frames 600. Start and end frames 604 of sip events are collected and next sip events 606 are retrieved. With each new sip event, the action unit, gesture and mental state lists are all initialized to zero (i.e. we are resetting the person's facial activity and mental state with each sip). The next frames in the event are retrieved 612 and if there are no more frames 614 then the frames are analyzed for head and facial activity and mental states and stored in the action unit, gesture and mental state lists 616 to obtain the predicted affective state 618 and the next sip event 606 is retrieved. If there are more frames 614 then 620 the analyses are appended to the current action unit, gesture and mental state lists. If there are no more sip events 608, then 622 return SipEventAffectiveState. Here, videos with high inter-coder matching are used as training examples. The system processes the input video and logs the analysis results. The system calculates confidence of the machine. The method then extracts the lowest T % of data the machine is confident about, these are sent to one or more human coders for spot-checking. Inter-coder agreement between the coders, as well as between machine and coders is computed (e.g., Cohen's Kappa). The videos with majority agreement are used as training examples. The videos with low inter-coder agreement are flagged for system operator to look at it, and for (dis)confirmatory labeling from more coders. The current invention also includes a method for the use of identified head gestures and facial expressions to identify events of interest. In one embodiment, consumers, in a series of trials, are given a choice of two beverages to sip and then asked to answer some questions related to their sipping experience. One of the main events of interest is that of the sip, where consumer product researchers are interested in primarily analyzing the customer's facial expression leading up to and immediately after the sip. Manually tagging the video with sip events is a time and effort-consuming task; at least two or three coders are needed to establish inter-rater reliability. As with event detection in video in general, several challenges exist with regard to machine detection and recognition of sip events. First, a good definition of what constitute a sip event is needed that covers the different ways with which people sip and defines the beginning and end of an event. Secondly, detecting sip events involve the detection and recognition of the person's face, their head gestures and the progression of these gestures over time. Third, events are often multi-modal, requiring fusion of vision-based analysis with semantic information from the problem domain and other available contextual cues. Finally, the sipping videos are different than those of say surveillance or sports; there are typically fewer people in the video, the amount of information available besides the video is minimal, compared to sports where there's an audio-visual track and lots of annotations. Also the events are subtler and there is typically only one camera view that is static. The approach of the disclosed embodiments is hierarchical and combines machine perception namely probabilistic models of facial expressions and head gestures with top-down semantic knowledge of the events of interest. The hierarchical models goes from low-level inferences about the presence of a face in the video and the person's head gesture (e.g., persistent head turn to the left) to more abstract knowledge about the presence of a sip event in the video. This hierarchy of actions allows the disclosed embodiments to model the complexity inherent in the problem of an event, such as sip detection, namely the multiple definitions and scenarios of a sip, as well as the uncertainty of the actions, e.g., whether the person is turning their head towards the cup or simply talking to someone else. In addition, the disclosed embodiments use semantic information from the event logs to increase the accuracy of the system. In this embodiment, a sip is characterized by the person turning towards the cup, leaning forward to grab the cup and then drinking from the cup (or straw). Face tracking and head pose estimation are used to identify when the person is turning, followed by a head gesture recognition system that identifies only persistent head gestures using a networks of dynamic classifiers (hidden Markov models). At the topmost level we have devised a sip detection algorithm that for each frame analyzes the current head gesture, the status of the face tracker and the event log, which in combination provide significant information about the person's sipping actions. Referring also to FIG. 6, a method is also disclosed to use automated methods to detect events of interest such as for example sips in a beverage tasting study.

Described below is an exemplary algorithm used for sip detection. The exemplary algorithm is shown as an example of how head gestures and facial expressions may be used to identify events of interest (in the specific example described, the event may be a person taking a sip, though in alternate embodiments the event of interest may be of any desired kind) in a video. Semantically, a sip event consists of orienting towards the cup, picking the cup, taking a sip and returning the cup before turning back towards the laptop to answer some questions. The input to the topmost level of our sip detection methodology consists of the following. Gestures[0, . . . , I], the vector of I persistent head turns and tilts; (identified as described in the gestures section). Tracker[0, . . . , T], describes the status of the tracker (on or off) at each frame of the video 0<t<T, which is needed because the face tracker stops when the head yaw or roll exceeds 30 degrees, which typically happens in sip events. EstStartofSip, which denotes the time within each trial when the participant is told which beverage to take a sip of (note that this is logged by the application and not manually coded) this time is offset by a few seconds WaitTime to allow the participant to read the outcome and begin the sipping action. TurnDuration is the minimum duration of a persistent head gesture that indicates a sip. EstQuestionDuration is the average time it takes to answer the questions following a sip event. As may be realized, in alternate embodiments, any suitable algorithm may be used to identify the event of interest. FIG. 9 shows an example 750 of detecting a sip by finding the longest head yaw/roll gesture within a specified time frame. In the first case as can be seen in FIG. 9, gestures is parsed for a tilt or a turn event such that EstStartofSip elapses between the start and end frames of the gesture. In this case, the start and end frames of the sip correspond to that of the gesture. In FIG. 9, an example of sip detected is shown using a combination of event log heuristics as well as observed head yaw/roll gestures. At each frame 756, 758, 760, 762, 764, 766, 768, 770 if the tracker is on, the facial feature points and rectangle around the face are shown. For each row of frames, the recognized head yaws and rolls 772 are shown in the top chart 752, while the output of the sip detection algorithm 774 is shown in the bottom 754. FIG. 10 shows an example 780 of a sip detected by a temporal sequence of detecting a head yaw/roll gesture followed by the tracker turning off. At each frame 782-810 if the tracker is on, the facial feature points and rectangle around the face are shown. In the second case as can be seen in FIG. 10, if a head gesture Gestures[i] 812 that persists for TurnDuration ends before EstStartofSip is found, the status of the face tracker is checked. A sip is detected if the tracker was off for at least M frames following the end of Gestures[i]. The parameter M ensures that any case where the tracker is off for a short period of time is ignored. If the first two cases do not return a head gesture before or around EstStartofSip, the rest of the trial is searched for head turns and tilts. The tilt or turn with the longest duration is considered to be the sip 814. Here is shown an exemplary breakdown of the sip detection algorithm for each participant. Case 1 looks for head yaws and rolls around EstStartofSip and account for 45% of sip detection; Case 2 looks for a head yaw or roll followed by the tracker turning off, accounting for 25% of the sips; Case 3 looks for the longest duration of a sip and accounts for 30% of the sips. The exemplary algorithm is set forth below:

Algorithm 1 Sip detection algorithm.

Input: Tracker[0,...,T], head yaw/roll gestures Ges-

tures[0,...,I], EstStartofSip, TurnDuration, EstQues-
tionDuration

Output: Sips[0,...,J]

SipFound ← FALSE
for all Gestures[i] from 0 to I do

if (Gestures[i].start <= EstStartofSip <= Ges-
tures[I].end) then

Sips[j].start ← Gestures[i].start
Sips[j].end ← Gestures[i].end
SipFound ← TRUE

end if

end for
if SipFound then

for all Gestures[i] from 0 to I do

if (Gestures[i].end <= EstStartofSip) and
(Gestures[i].duration > TurnDuration) and
(Tracker[t]=0) then

Sips[j].start ← Gestures[i].start
Sips[j].end ← Gestures[i].end
SipFound ← TRUE

end if

end for

end if
if SipFound then

G ← GetLongest(Gestures[0,...,I])
Sips[j].start ← G.start
Sips[j].start ← G.end

end if

As noted before, the above noted algorithm is merely exemplary and provided herein to assist the description of the exemplary embodiments. As may be realized, in alternate embodiments any other suitable algorithm may be used. Referring now to FIG. 11, there is shown an example embodiment 830 of feature point locations 6-24 that are tracked and represented. Feature points represented by a star 23, 24, A are extrapolated.

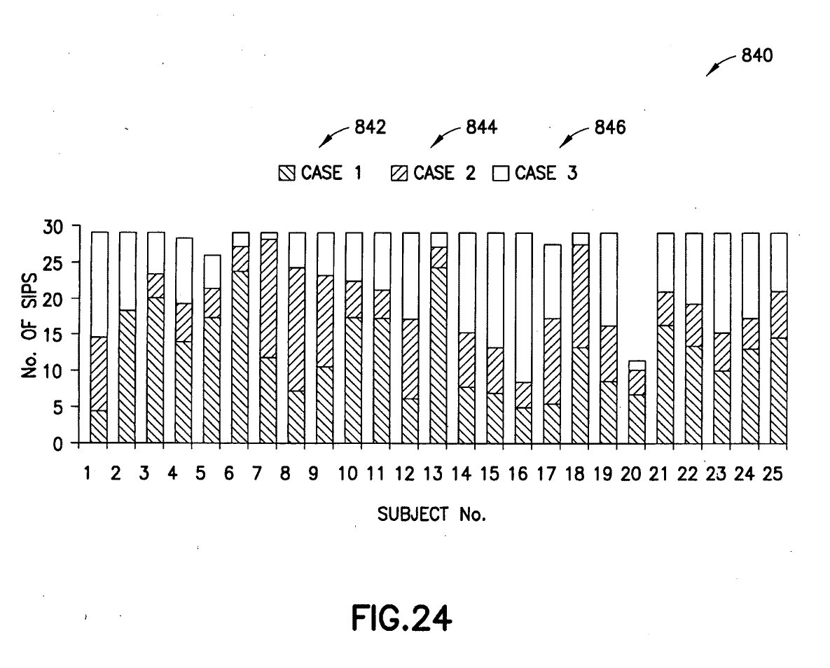

Referring now to FIG. 24, there is shown an exemplary distribution 840 of cases of sips (as noted previously, though the exemplary embodiment is described with specific reference to sip events as the events of interest, in alternate embodiments the events of interest may be of any other desired kind) for each participant in an example corpus. Case 1 842 accounts for 45% of the detected sips; case 2 844 accounts for 25%, while case 3 846 accounts for the remaining 30% of sips. The algorithm above only deals with a single sip per trial. However, the participants often chewed or drank water before taking a sip of the beverage. Thus, any number of sips could occur within EstStartofSip right up to EstQuestionDuration before the start of the next trial, which is the time it takes the participant to answer questions related to their sipping experience. To handle multiple sips within a trial, persistent head gestures that: (1) occur after EstStartofSip; (2) start within EstQuestionDuration before the start of the next trial and (3) last for at least TurnDuration are all returned as possible sips. The methodology successfully detects single and multiple sips in over 700 examples of sip events with an average accuracy, for example, of 78%. Again, this system and method is not limited to the detection of sipping events. It can be applied, for example, to other events capable of being detected such as from facial expression and/or head gesture sequences.

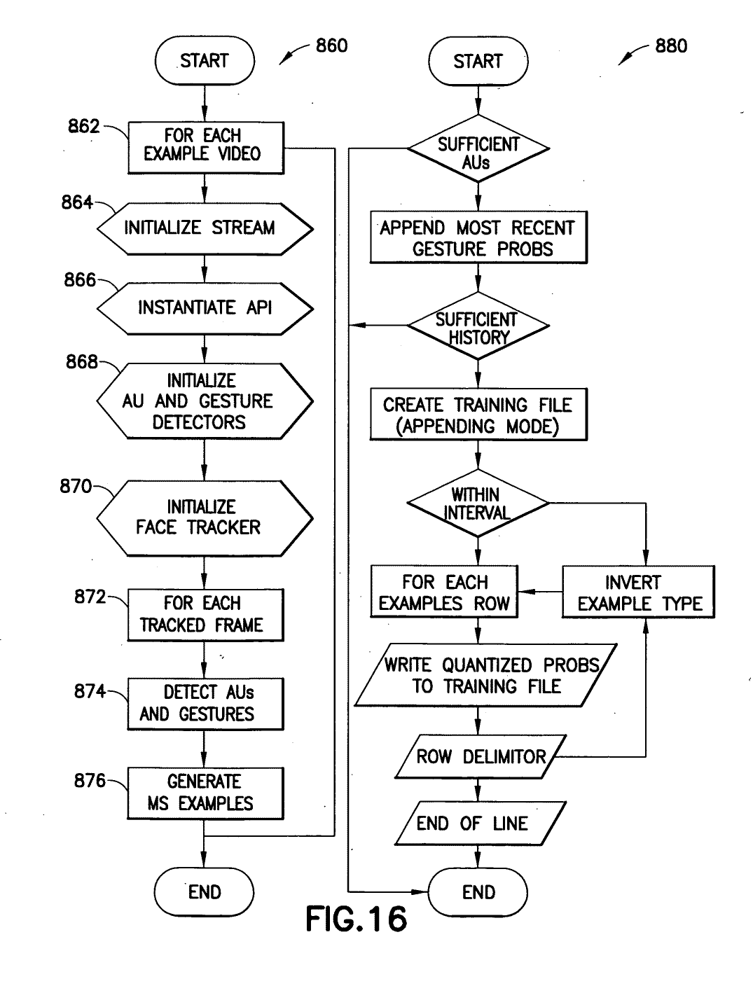

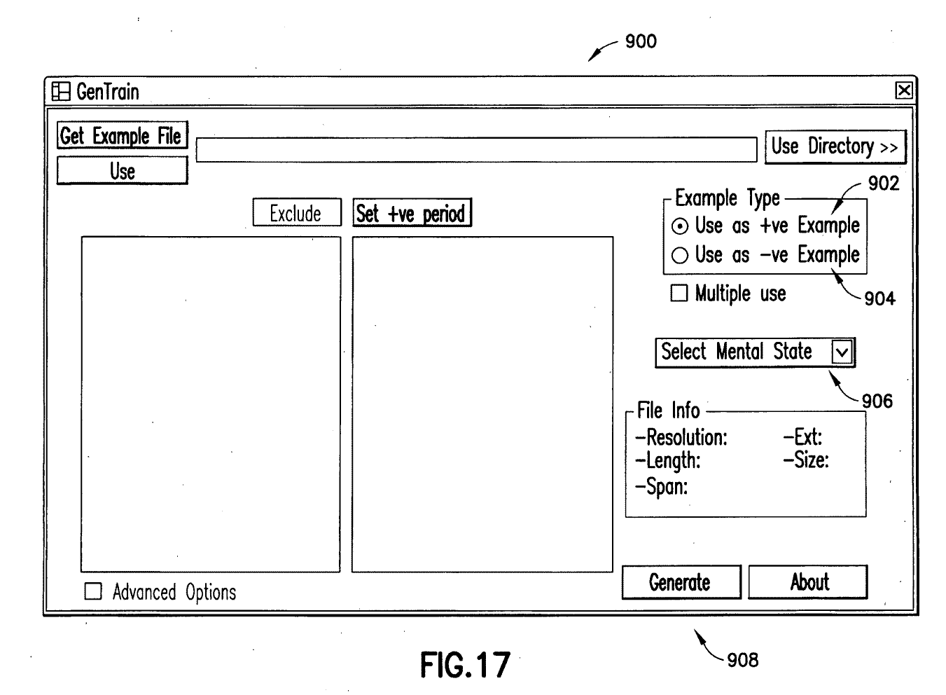

Referring now to FIGS. 16 and 17, there is shown training and re-training of gesture and mental state classifiers. Here, FIG. 16 is a flowchart showing the general steps involved in retraining existing gestures or adding new gestures to the system where the flowchart shows training and retraining of mental states. The method is data-driven, meaning that gesture and/or mental state classifiers can be (re)trained provided that there are video examples of these states to provide to the system. Here, the apparatus can be easily adapted to new applications, cultures, and domains, e.g. in cultures where head nods and shakes may have different meaning, or in domains such as business where expressions may be less subtle, or in a specific application where very specific expressions are of interest and the system is tuned to focus on this subset. To retrain an existing mental state classifier or train a new mental state classifier, M video clips representative of the mental state are selected, these M clips show one or more persons expressing the mental state of interest through their face and head movements. These M clips represent the positive training set for the process. N video clips representative of one of more persons expressing other mental states through face and head, movements are also selected. These N clips represent the negative training set for the process. (A video may contain one or more overlapping or discontinuous segments that constitute the positive examples, while the rest would constitute negative examples; the method presented herein allows for specific intervals of a video clip to be used as positive, and the rest as negative). The system 860 is then run in training mode where M+N clips are processed to generate a list of training examples as follows. For each video 862, the relevant subinterval is loaded. The stream 864, API 866, face tracker 870, ActionUnit and Gesture modules 868 are initialized. Then for each frame where a face is found 872, the action unit and gesture classifiers 874 are invoked. In one embodiment of the system, the gestures are quantized to binary values. They are then logged into an “evidence” array of a pre-defined size (6 in one case) of gestures. Each row of the training file represents one training example: the first column indicates whether the example is a positive or negative one, the next set of columns show examples of gestures. Once this file is complete, the mental state inference engine is invoked with the training file 876. It then iterates through the examples until it converges on the parameters. An .xml file representing the mental classifiers is produced. If an existing mental state is being re-training, the XML file replaces the current one. The procedure for retraining 880 an existing mental state and training to introduce a new mental state to the system may be identical. FIG. 17 shows a snapshot of the user interface 900 used for training mental states. A set of videos are designated as positive examples 902 of a mental state; and another set of videos are designated as the negative examples 904. A mental state 906 is selected. Then the training function is invoked 908. The training function generates training examples for each mental state and creates a new XML file for the mental state.

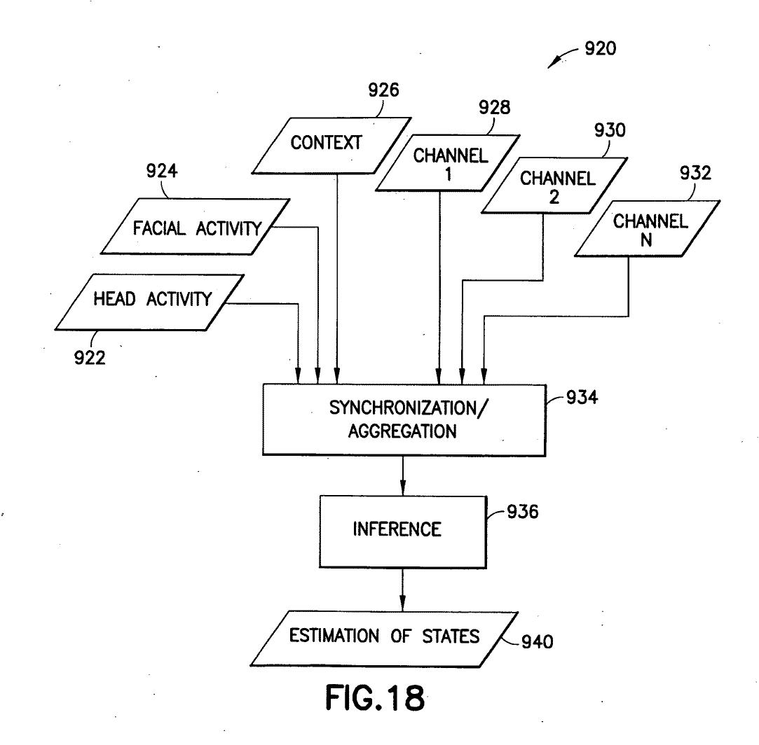

Referring now to FIG. 18, multi-modal analysis 920 is shown where FIG. 18 shows a flowchart depicting multi-modal analysis. Head 922 and facial 924 activity is analyzed and recorded along with contextual information 926, and additional channels of information 928, 930, 932 such as physiology (skin conductance, motion, temperature). This data is synchronized and aggregated 934 over time, and input to an inference engine 936 which outputs a probability for a set of affective and cognitive states 940. Here, the disclosed embodiments includes a method and system for multi-modal analysis. In one embodiment of the system, the apparatus, which consists of a video camera that records head and facial activity, is used in a multi-modal setup jointly with other sensors microphones to record the person's speech, video camera to track a person's body movements, physiology sensors to monitor skin conductance, heart rate, heart rate variability and other sensors (e.g., motion, respiration, eye-tracking, etc). Contextual information including but not limited to task information and setting is also recorded. For example, in an advertisement viewing scenario, head yaw events separate frontal video clips from non-frontal ones where the customer turned his or face away from the advertisement; in a usability study for tax software, head yaws signal that the person is turning to the side to check physical documents; in a sipping study head yaws signal turning to possibly engage with the product placed to the side of the computer/camera. A method is applied to synchronize the various channels of information and aggregate the incoming data. Once synchronized the information is passed onto multiple affective and cognitive state classifiers for inference of the states. This method enhances confidence of an interpretation of a person's state and extends the range of states that can be inferred. An action handler is also provided. Here, a number of action and reporting options exist for representing the output of the system. Such options include specifically, but not exclusively, (i) a combination of log files at each level of analysis for each frame of the video for each individual; (ii) graphical visualization of the data at each level of analysis for each frame of the video; (iii) an aggregate compilation of the data across multiple levels across multiple persons.

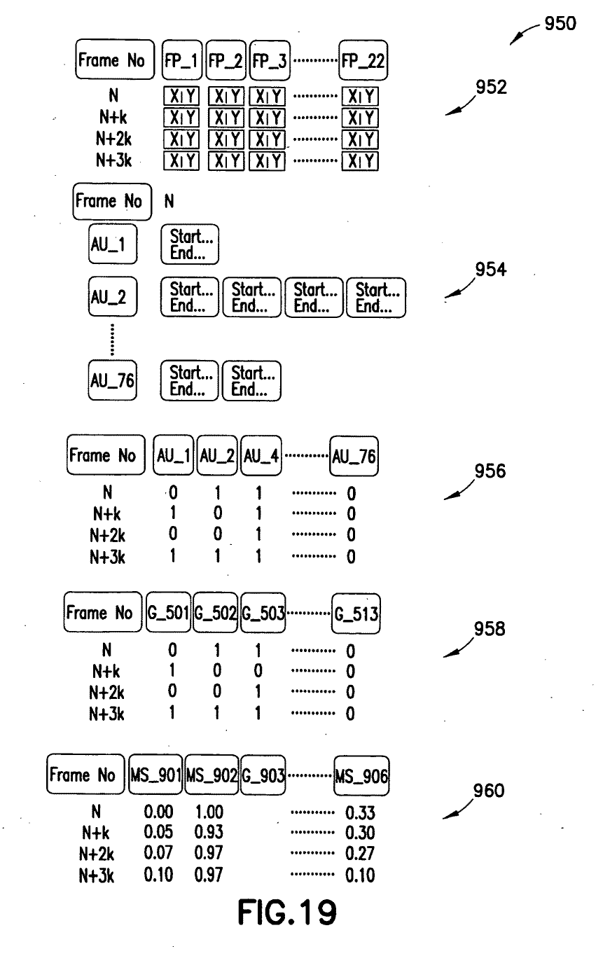

Referring now to FIG. 19, log files 950 are shown. Here, the disclosed embodiments include log functions that write the data stored in all the buffers to text files. Here, events or interactions logged, tagged and linked or correlated to inferred states. The output of first stage of analysis consists of multiple logs. The Face Tracker log 952 has a vector of the face tracker's status Tracker[0, . . . , T], where at frame t, Tracker[t] is either on (a value of 1) or off (a value of 0) indicating whether a face was found or not. The ActionUnit log 954 includes a line for each action unit for each frame; each line contains the Action Unit name and the number of instances detected of this Action Unit and the length of each instance (start frame and End Frame), so it is essentially a memory dump of the action unit buffer; alternatively, the ActionUnit log file 956 may be structured to only show the action units detected per frame. The latter lends itself to graphical output. The Gesture log 958 has where each column represent the Gestures and the rows represent the frame numbers at which the detect function was invoked. Each cell contains the raw probability output by the classifier. An alternate structure depicts either 1 or 0 depending on whether or not the gesture was detected in that frame number, according to a preset threshold. For instance, a threshold of 0.4 would mean that any probability below or equal to 0.6 will be quantized to 0, and any probability greater than 0.4 will be quantized to 1. The Mental State log 960 is similar to the Gesture log, but the columns represent the mental states and the rows represent the frame numbers at which the function detect Mental States( ) was invoked. Each cell contains the raw probability output by the classifier. An alternate structure for the log depicts either 1 or 0 depending on whether or not the mental state was detected in that frame number, according to a preset number. For instance, a threshold of 0.4 would mean that any probability below or equal to 0.6 will be quantized to 0, and any probability greater than 0.4 will be quantized to 1. In addition, an example below that demonstrates how events are correlated to inferred states where the example builds on the sip detection example. Here, when gestures are used to infer an event (e.g., whether the person is sipping a beverage), these events are time stamped and typically the onset of the event and offset is inferred, for example, the length of sip based on information from the gesture buffer as well as the interaction context, for example, average length of sips. In another example, when a group of people are watching a movie trailer or movie clip, the resulting facial video is time synced with the video frames, and observed facial and head activity or inferred mental states may be synchronized to events in the video.

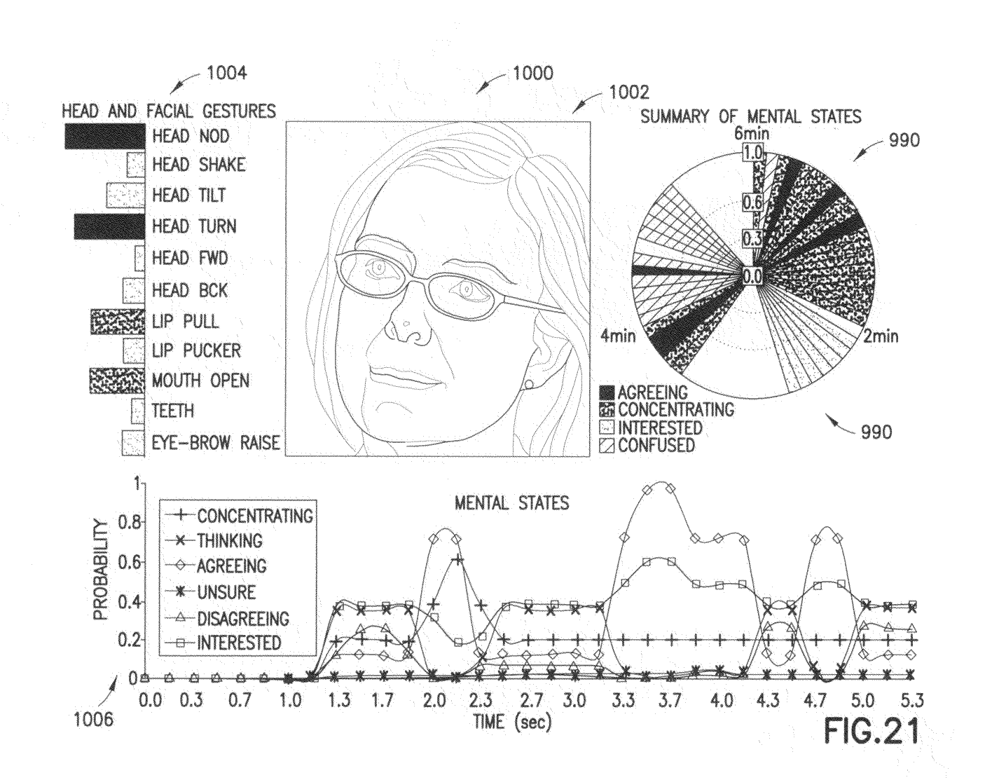

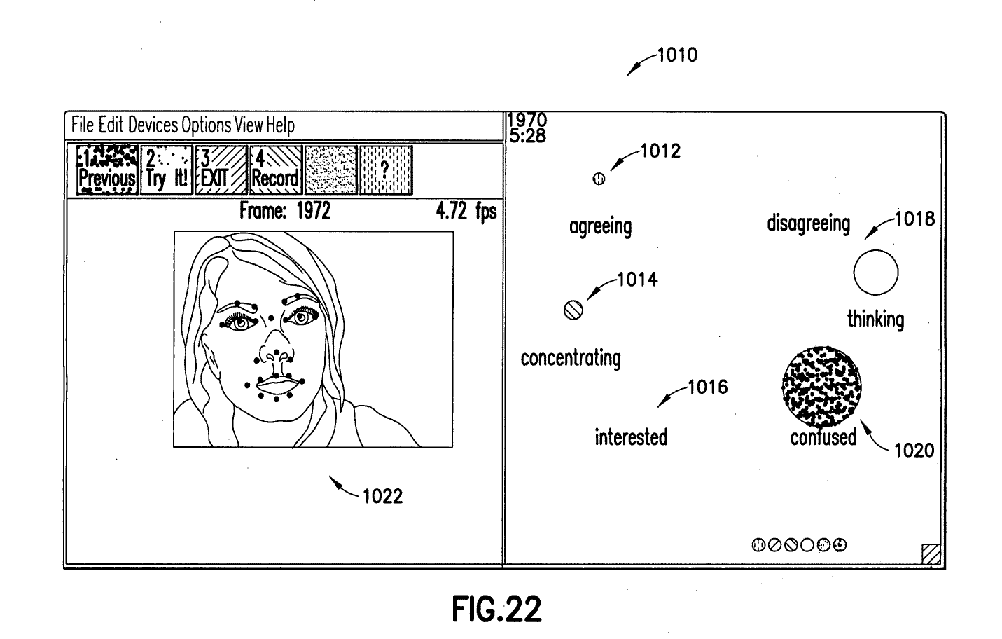

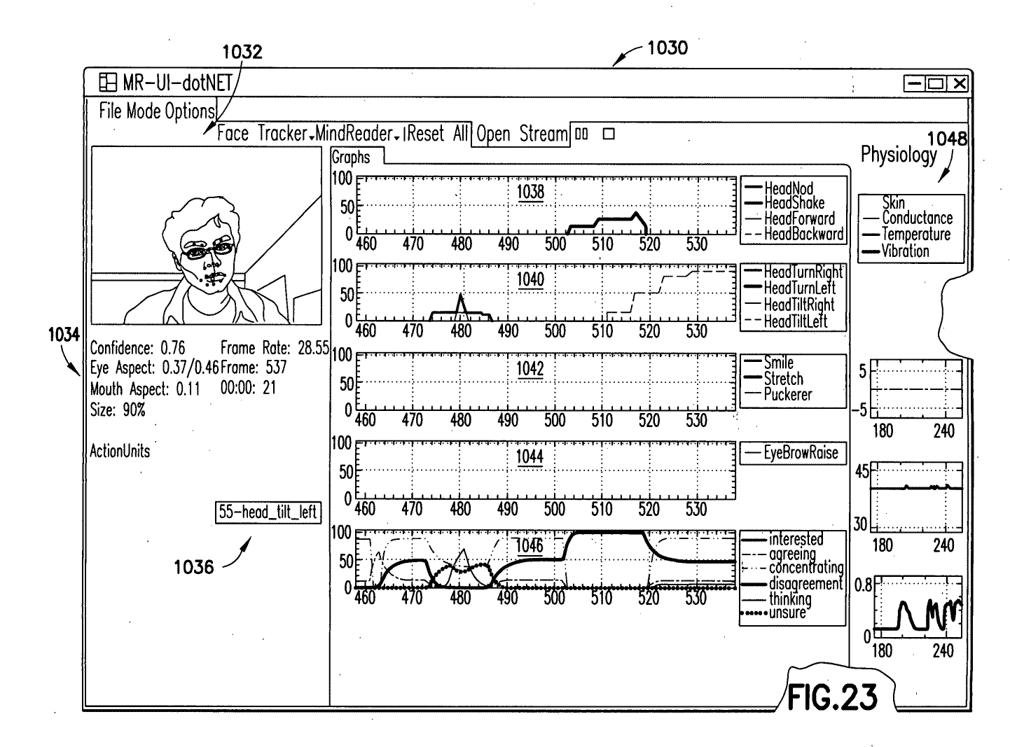

Referring now to FIG. 20, graphical visualization 970 is shown. Here FIGS. 20-23 show a snapshot of the head and facial analysis system and the plots that are output. In FIG. 20, on the upper left of the screen, the person's video 972 is shown along with the feature point locations. Below the frame 974 is information relating to the confidence of the face finder, the frame rate, the current frame being displayed, as well as eye aspect ratio and face size. On the lower left 976, the currently recognized facial and head action units are highlighted. The line graphs on the right show the probabilities of the various head gestures 978, 980, facial expressions 982, 986 as well as mental states 984. Several options may be implemented for the visual output of the disclosed embodiments. The graphical visualizations can be organized by a number of factors: (1) which level of information is being communicated (face bounding box, feature point locations, action units, gestures, and mental states); (2) the degree of temporal information provided. This ranges from no temporal information, where the graph provides a static snapshot of what is detected at a specific point in time (e.g., bar charts in FIG. 20, showing the gestures at a certain point in time), to views that offer temporal information or history (e.g., radial chart 990 in FIG. 21, showing history of a person's over an extended period of time); (3) the window size and sliding factor. In FIG. 20, there is shown a snapshot of one visual output of head and facial analysis system and the plots that are output. On the upper left of the screen the person's video is shown along with the feature point locations. Below the frame is information relating to the confidence of the face finder, the frame rate, the current frame being displayed, as well as eye aspect ratio and face size. On the lower left, the currently recognized facial and head action units are highlighted. The line graphs on the right show the probabilities of the various head gestures, facial expressions as well as mental states. FIG. 25 shows different graphical output given by the system 1000, including a radial chart 990. In the center, the person's video 1002 is shown. In FIG. 21, there is shown another possible output of the system being a radial view that shows the person's most likely mental state over an extended period of time, giving a bird's eye view or general sentiment of a person's state. The probability of the head gestures and facial expressions are displayed as bar graphs 1004 on the left; the bar graphs are color coded to displayed a high likelihood or confidence that the gesture is observed on the person's face. The line graphs 1006 on the bottom show the probability of the mental states over time. The graphs are dynamic and move as the video moves. On the right, a radial chart 990 summarizes the most likely mental state at any point in time. FIG. 22 shows instantaneous output 1010 of just the mental state levels, shown as bubbles 1012, 1014, 1016, 1018, 1020 that increase in radius (proportional of probability) depending on the mental state, for example agreeing, disagreeing, concentrating, thinking interested or confused. The person's face 1022 is shown to the left, with the main facial feature points highlighted on the face. In FIG. 26, there is shown instantaneous output of just the mental state levels at any point in time. The person's face is shown to the left, with the main facial feature points highlighted on the face. The probability of each gesture and/or mental state is mapped to the radius of a bubble/circle, called an Emotion Bubble, which is computed as a percentage of a maximum radius size. This interface was specifically designed to provide information about current levels of emotions or mental states in a simple and intuitive way that would be easily accessible to individuals who have cognitive difficulties (such as those diagnosed with an autism spectrum disorder), without overloading the output with history. The system is customizable by individual users, letting users choose how emotions are represented by varying factors such as colors of the Emotion Bubbles or the line graphs; font size of labels underneath the Emotion Bubbles; position of the Emotion Bubbles; and background color behind the Emotion Bubbles. By allowing users easy access to the parameters that characterize the interface, the system allows users to change the interface in order to increase their own comfort level with its display. In this embodiment the colors where chosen so that the “positive” emotions are assigned “cool” colors (green, blue, and purple) indicating a productive state, and the “negative emotions” are assigned “warm” colors (red, orange, and yellow) indicating that the user of the interface should be aware of a possible conversational impediment. FIG. 23 shows multi-modal analysis 1030 showing facial and head events as well as physiological signals (temperature, electrodermal activity and motion). Snapshot of the head and facial analysis system and the plots that are output. On the upper left of the screen the person's video 1032 is shown along with the feature point locations. Below the frame 1034 is information relating to the confidence of the face finder, the frame rate, the current frame being displayed, as well as eye aspect ratio and face size. On the lower left 1036, the currently recognized facial and head action units are highlighted. The line graphs on the right 1038, 1040, 1042, 1044, 1046 show the probabilities of the various head gestures, facial expressions as well as mental states. On the rightmost column 1048, physiological signals are plotted and synchronized with the facial information. In FIG. 27, there is shown multi-modal analysis of facial and head events as well as physiological signals (temperature, electrodermal activity and motion). Here, there is shown a snapshot of the head and facial analysis system and the plots that are output. On the upper left of the screen the person's video is shown along with the feature point locations. Below the frame is information relating to the confidence of the face finder, the frame rate, the current frame being displayed, as well as eye aspect ratio and face size. On the lower left, the currently recognized facial and head action units are highlighted. The line graphs on the right show the probabilities of the various head gestures, facial expressions as well as mental states. On the rightmost column, physiological signals are plotted and synchronized with the facial information.

Light, Audio and Tactile Output are also provided for where the disclosed embodiments include a method for computing the best point in time to give a form of feedback to one or more persons in real-time. The possible feedback mechanisms include light (e.g., in the form of LED feedback mounted on a wearable camera or eyeglasses frame), audio, or vibration output. After every video frame is processed, the probabilities of the mental states are checked, and if a mental state probability stays above the predefined maximum threshold for a defined period of time, it gets marked as the current mental state and its corresponding output (e.g., sound file) is triggered. The mental state stays marked until its probability decrease below the predefined minimum threshold.

The disclosed apparatus may have many different embodiments. A first embodiment applies to advertising and marketing. Here, the apparatus yields tags that at the top-most level describe the interest and excitement levels individuals or groups have about a new advertisement or product. For example, people could watch ads on a screen (small phone screen or larger display) with a tiny camera pointed at them, which labels things such as how often they appeared delighted, annoyed, bored, confused, etc. A second embodiment applies to product evaluation, including usability. Here, customers are asked to try out a new product (which could be a new gadget, a new toy, a new beverage or food, a new automobile dashboard, a new software tool, etc) and a small camera is positioned to capture their facial-head movements during the interactive experience. The apparatus yields tags that describe liking and disliking, confusion, or other states of interest for inferring where the product use experience could be improved. A third embodiment applies to customer service. Here, the technology is embedded in ongoing service interactions, especially online services, ATM's, as well as face-to-face encounters with software agents, human or robotic customer service representatives, to help automate the monitoring of expressive states that a person would usually monitor for improving the service experience. A fourth embodiment applies to social cognition understanding. Here, the technology provides a new tool to quantitatively measure aspects of face-face social interactions including synchronization and empathy. A fifth embodiment applies to learning. Here, in distance learning and other technology-mediated learning scenarios (e.g. electronic piano tutor, training of facial control for negotiations, therapy, or poker-playing sessions) the technology can measure engagement, states of flow and interest as well as boredom, confusion, and frustration, and adapt the learning experience accordingly to maximize the student's interest. A sixth embodiment applies to cognitive load measures. Here, in tasks including driving, air traffic control, and operation of dangerous machinery or facilities, the technology can visually detect signs related to cognitive overload. When the facial-head expressive patterns are combined with other channels of information (e.g. heart-rate variability, electrodermal activity) this can build a more confident measure of the operator's state. A seventh embodiment applies to a social training tool. Here, the technology assists with functions like reading and understanding facial expressions of oneself and others, initiating conversation, taking turns during conversation, gauging the listener's level of interest and mental state, mirroring, help with responding with empathic nonverbal cues, and help on deciding when to pause and/or end a conversation. This is helpful for marketing/salesperson training as well as for persons with social difficulties. A seventh embodiment applies to epilepsy analysis. Here, the system measures facial expressions prior to and during epileptic seizures, for characterization and prediction of the ictal onset zone, thereby providing additional evidence information in the presurgical and diagnostic workup of epilepsy patients. The invention can be used to infer whether any of the observed lateralizing ictal features can be detected prior to or at the start of an epileptic seizure and therefore can predict or detect seizures non-invasively.

It should be understood that the foregoing description is only illustrative of the invention. Various alternatives and modifications can be devised by those skilled in the art without departing from the invention. Accordingly, the present invention is intended to embrace all such alternatives, modifications and variances which fall within the scope of the appended claims.

What is claimed is:

1. A method comprising:
a digital computer:
processing data indicative of images of facial and head movements of a subject to recognize at least one of said movements and to determine at least one mental state of said subject,
outputting instructions for providing to a user information relating to at least one said mental state, and
further processing data reflective of input from a user, and based at least in part on said input, confirming or modifying said determination, and
generating with a transducer an output of humanly perceptible stimuli indicative of said at least one mental state.

2. The method of claim 1, wherein processing with said computer comprises calculating a value indicative of certainty, or of a range of certainties, or of probability, or of a range of probabilities, in case regarding said at least one mental state.

3. The method of claim 1, wherein outputting instructions comprises providing to a user substantially real time information regarding said at least one mental state.

4. The method of claim 3, wherein said computer is adapted to recognize a set of the mental states that comprises at least seven elements, at least one of which is a mental state other than a “basic emotions” of happiness, sadness, anger, fear, surprise and disgust.

5. The method of claim 3, wherein said computer is adapted to recognize at least two types of events, and wherein at least one said type of event has a shorter time duration than at least one other said type of event.

6. The method of claim 3, wherein said computer is adapted to recognize facial or head movements that are asynchronous or overlapping.

7. The method of claim 3, wherein a plurality of recognized facial or head movements is mapped to a single mental state.

8. The method of claim 1, wherein at least one said transducer comprises a graphical user interface.

9. The method of claim 1, wherein outputting instructions with said computer comprises providing to the user one or more images of said facial or head movements and substantially concurrently providing to said user information regarding said at least one mental state associated with said movements.

10. The method of claim 1, wherein processing includes using data consciously inputted or provided by said subject.

11. The method of claim 1, wherein processing includes using physiological data regarding said subject.

12. The method of claim 1, wherein at least part of said computer is remote from said user.

13. The method of claim 1, wherein outputting instructions comprises providing to a user a summary of mental states inferred from facial and head movements over a period of time.

14. The method of claim 1, further comprising associating, with said computer, said at least one mental state with at least two events, wherein at least one of said events is indicated by said data indicative of images of facial and head movements and wherein at least one other of said events is indicated by another data set, which other data set comprises content provided to said subject or data recorded about said subject.

15. The method of claim 1, further comprising processing data indicative of images of facial and head movements of a plurality of subjects to determine mental states of the plurality of subjects.

16. The method of claim 3, wherein said real time information is provided to a plurality of, users and input from a plurality of users is processed.

17. A method comprising:
with a digital computer:
processing data indicative of images of facial and head movements of a subject to determine at least one mental state of said subject, and
associating said at least one mental state with at least two events, wherein at least one of said events is indicated by said data indicate of images of facial and head movements and wherein at least one other of said events is indicated by another data set, which other data set comprises content provided to said subject or data recorded about said subject.

18. The method of claim 17, wherein said association employs at least one time stamp, frame number or other value indicative of temporal order.

19. The method of claim 17, wherein said content provided to said subject comprises the display of an audio or visual content.

20. The method of claim 17, wherein processing comprise processing physiologic data recorded about said subject.

21. The method of claim 17, wherein processing comprises processing data recorded relating to said subject's interaction with a graphical user interface.

22. The method of claim 17, further comprising, with said computer outputting instructions providing to a user substantially real time information relating to said at least one mental state.

23. The method of claim 17, further comprising, with said computer analyzing data reflective of input from a user, and based at least in part on said analysis of said input, to change or confirm at least one said determination.

24. An apparatus comprising:
at least one camera for capturing images of facial and head movements of a subject; and
at least one computer adapted for:
analyzing data indicative of said images and determining one or more mental states of said subject,
outputting digital instructions for providing a user substantially real time information relating to said at least one mental state,
analyzing data reflective of input from a user, and based at least in part on said user input data analysis, changing or confirming said determination.

25. An article of manufacture, comprising a machine-accessible medium having instructions encoded thereon for enabling a computer to perform the operations of:
processing data indicative of images of facial and head movements of a subject to recognize at least one said movement and to determine at least one mental state of said subject,
outputting instructions for providing to a user information relating to said at least one mental state, and
processing data reflective of input from a user, and based in least in part on said input, confirm or modify said determination.
